SEQUENCE LISTING (93 sequences)

SEQ ID NO: 1 - protein, 7 aa, Homo sapiens
RPRTSSF

SEQ ID NO: 2 - protein, 8 aa, Artificial
  Synthetic peptide
  MISC_FEATURE (1)..(1): Xaa is absent or is selected from DLys4 or Lys4; some residues may be missing.
  MISC_FEATURE (2)..(2): Xaa = Arg, Lys, Orn or Dab
  MISC_FEATURE (5)..(5): Xaa = Nva, Leu, Ile, Abu or Orn
  MISC_FEATURE (6)..(6): Xaa = Tyr, Gly, GlyNH2, Ser(OMe), Glu, or Glu (CO-N-SO2-isoquinoline)
  MISC_FEATURE (7)..(7): Xaa = Dap, Abu, GlyNH2, Ser(OMe), Gly, Ala or Ser
  MISC_FEATURE (8)..(8): Xaa = aromatic or an aliphatic bulky residue
XXPRXXXX

SEQ ID NO: 3 - protein, 8 aa, Artificial
  Synthetic peptide
  MISC_FEATURE (1)..(1): Xaa is absent or is selected from DLys2-4 or Lys2-4; some residues may be missing.
  MISC_FEATURE (5)..(5): Xaa = Nva, Leu, Ile, Abu or Orn
  MISC_FEATURE (6)..(6): Xaa = Tyr, Gly, GlyNH2, Ser(Me), Glu, or Glu (NH-(CH2)2-NH-SO2-iso quinoline)
  MISC_FEATURE (7)..(7): Xaa = Dap, Abu, GlyNH2, Ser(Me), Gly, Ala or Ser
  MISC_FEATURE (8)..(8): Xaa = an aromatic or an aliphatic bulky residue
XRPRXXXX

SEQ ID NO: 4 - protein, 8 aa, Artificial
  Synthetic peptide
  MISC_FEATURE (1)..(1): Xaa = a spacer amino acid or an amino acid that is absent
  MISC_FEATURE (5)..(5): Xaa = Norvaline
  MISC_FEATURE (7)..(7): Xaa = Dap or Ala
  MISC_FEATURE (8)..(8): Xaa = Homoleucine (Hol)
XRPRXYXX

SEQ ID NO: 5 - protein, 10 aa, Artificial
  Synthetic peptide
  MISC_FEATURE (1)..(1): DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
KKKRPRXYXX

SEQ ID NO: 6 - protein, 10 aa, Artificial
  Synthetic peptide
  MISC_FEATURE (7)..(7): Xaa=Nva
  MISC_FEATURE (9)..(9): Xaa=Dap
  MISC_FEATURE (10)..(10): Xaa=Homoleucine
KKKRPRXYXX

SEQ ID NO: 7 - protein, 7 aa, Artificial
  Synthetic peptide
  MISC_FEATURE (4)..(4): Xaa=Nva
  MISC_FEATURE (6)..(6): Xaa=Dap
  MISC_FEATURE (7)..(7): Xaa=Homoleucine
RPRXYXX

SEQ ID NO: 8 - protein, 7 aa, Artificial
  Synthetic peptide
  MISC_FEATURE (4)..(4): Xaa=Orn
  MISC_FEATURE (5)..(5): Xaa = Glu-(CO-N-SO2-Isoquinoline)
RPRXXSF

SEQ ID NO: 9 - protein, 7 aa, Artificial
  Synthetic peptide
  MISC_FEATURE (4)..(4): Xaa=Nva
  MISC_FEATURE (7)..(7): Xaa=Homoleucine
RPRXYAX

SEQ ID NO: 10 - protein, 10 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 11 - protein, 10 aa, Artificial
  Synthetic peptide
  MISC_FEATURE (1)..(1): Xaa=Cholesteryl-O-CO-Lys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 12 - protein, 11 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (4)..(4): DLys
  MISC_FEATURE (8)..(8): Xaa=Nva
  MISC_FEATURE (10)..(10): Xaa=Dap
  MISC_FEATURE (11)..(11): Xaa=Homoleucine
  MOD_RES (11)..(11): AMIDATION
XKKKRPRXYXX

SEQ ID NO: 13 - protein, 13 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (10)..(10): Xaa=Nva
  MISC_FEATURE (12)..(12): Xaa=Ser(Me)
  MISC_FEATURE (13)..(13): Xaa=Homoleucine
  MOD_RES (13)..(13): AMIDATION
XKKKKKRPRXYXX

SEQ ID NO: 14 - protein, 10 aa, Artificial
  Synthetic peptide
  MISC_FEATURE (1)..(1): Xaa=Cholesteryl-O-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MISC_FEATURE (8)..(8): DLys
  MISC_FEATURE (9)..(9): DLys
  MISC_FEATURE (10)..(10): DLys
  MOD_RES (10)..(10): AMIDATION
XPRXYXXKKK

SEQ ID NO: 15 - protein, 10 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Ser(Me)
  MISC_FEATURE (10)..(10): Xaa=Homoleucine
  MOD_RES (10)..(10): AMIDATION
XKKRPRXYXX

SEQ ID NO: 16 - protein, 10 aa, Artificial
  Synthetic peptide
  MISC_FEATURE (1)..(1): Xaa=Cholesteryl-O-CO-Lys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): C terminal - OH
XKKRPRXYXX

SEQ ID NO: 17 - protein, 10 aa, Artificial
  Synthetic peptide
  MISC_FEATURE (1)..(1): Xaa=Cholesteryl-O-CO-(CH2)2-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 18 - protein, 7 aa, Artificial
  Synthetic peptide
  MISC_FEATURE (1)..(1): Xaa=Cholesteryl-O-CO-Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 19 - protein, 10 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKKPRXYXX

SEQ ID NO: 20 - protein, 10 aa, Artificial
  Synthetic peptide
  MISC_FEATURE (1)..(1): Xaa=Vitamin E-CO-(CH2)2-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 21 - protein, 9 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (6)..(6): Xaa=Nva
  MISC_FEATURE (8)..(8): Xaa=Dap
  MISC_FEATURE (9)..(9): Xaa=Homoleucine
  MOD_RES (9)..(9): AMIDATION
XKRPRXYXX

SEQ ID NO: 22 - protein, 7 aa, Artificial
  Synthetic peptide
  MISC_FEATURE (1)..(1): Xaa=Cholesteryl-O-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 23 - protein, 7 aa, Artificial
  Synthetic peptide
  MISC_FEATURE (1)..(1): Xaa=Cholesteryl-O-CO-Arg
  MISC_FEATURE (4)..(4): Xaa=Nva
  MISC_FEATURE (7)..(7): Xaa=Homoleucine
  MOD_RES (7)..(7): AMIDATION
XPRXYAX

SEQ ID NO: 24 - protein, 8 aa, Artificial
  Synthetic peptide
  MISC_FEATURE (1)..(1): Xaa=Cholesteryl-O-CO-Gly
  MISC_FEATURE (5)..(5): Xaa=Nva
  MISC_FEATURE (8)..(8): Xaa=Homoleucine
  MOD_RES (8)..(8): AMIDATION
XRPRXYAX

SEQ ID NO: 25 - protein, 7 aa, Artificial
  Synthetic peptide
  MISC_FEATURE (1)..(1): Xaa=Vitamin E-Succinate-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 26 - protein, 17 aa, Artificial
  Synthetic peptide
  MISC_FEATURE (1)..(1): Xaa=DArg
  MISC_FEATURE (2)..(2): Xaa=DArg
  MISC_FEATURE (3)..(3): Xaa=DArg
  MISC_FEATURE (4)..(4): Xaa=DArg
  MISC_FEATURE (5)..(5): Xaa=DArg
  MISC_FEATURE (6)..(6): Xaa=DArg
  MISC_FEATURE (7)..(7): Xaa=DArg
  MISC_FEATURE (8)..(8): Xaa=DArg
  MISC_FEATURE (9)..(9): Xaa=DArg
  MISC_FEATURE (14)..(14): Xaa=Nva
  MISC_FEATURE (17)..(17): Xaa=Homoleucine
  MOD_RES (17)..(17): AMIDATION
XXXXXXXXXGRPRXYAX

SEQ ID NO: 27 - protein, 7 aa, Artificial
  Synthetic peptide
  MISC_FEATURE (1)..(1): Xaa=Cholesteryl-O-CO-Arg
  MISC_FEATURE (4)..(4): Xaa=Orn
  MISC_FEATURE (5)..(5): Xaa=Glu(Aminoethylsulfonamidoisoquinoline)
  MOD_RES (7)..(7): AMIDATION
XPRXXSF

SEQ ID NO: 28 - protein, 8 aa, Artificial
  Synthetic peptide
  MISC_FEATURE (1)..(1): Xaa=Myristyl-Gly
  MISC_FEATURE (5)..(5): Xaa=Nva
  MISC_FEATURE (7)..(7): Xaa=Dap
  MISC_FEATURE (8)..(8): Xaa=Homoleucine
  MOD_RES (8)..(8): AMIDATION
XRPRXYXX

SEQ ID NO: 29 - protein, 23 aa, Artificial
  Synthetic peptide
  MISC_FEATURE (20)..(20): Xaa=Nva
  MISC_FEATURE (22)..(22): Xaa=Dap
  MISC_FEATURE (23)..(23): Xaa=Homoleucine
RQIKIWFQNRRMKWKKRPRXYXX

SEQ ID NO: 30 - protein, 23 aa, Artificial
  Synthetic peptide
  MISC_FEATURE (20)..(20): Xaa=Nav
  MISC_FEATURE (23)..(23): Xaa=Homoleucine
RQIKIWFQNRRMKWKKRPRXYAX

SEQ ID NO: 31 - protein, 17 aa, Artificial
  Synthetic peptide
  MISC_FEATURE (4)..(4): Xaa=Nva
  MISC_FEATURE (6)..(6): Xaa=Dap
  MISC_FEATURE (7)..(7): Xaa=Homoleucine
  MISC_FEATURE (8)..(8): DLys
  MISC_FEATURE (9)..(9): DLys
  MISC_FEATURE (10)..(10): DLys
  MISC_FEATURE (11)..(11): DLys
  MISC_FEATURE (12)..(12): DLys
  MISC_FEATURE (13)..(13): DLys
  MISC_FEATURE (14)..(14): DLys
  MISC_FEATURE (15)..(15): DLys
  MISC_FEATURE (16)..(16): DLys
  MISC_FEATURE (17)..(17): DLys
RPRXYXXKKKKKKKKKK

SEQ ID NO: 32 - protein, 15 aa, Artificial
  Synthetic peptide
  MISC_FEATURE (1)..(1): DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (7)..(7): DLys
  MISC_FEATURE (8)..(8): DLys
  MISC_FEATURE (12)..(12): Xaa=Nva
  MISC_FEATURE (14)..(14): Xaa=Dap
  MISC_FEATURE (15)..(15): Xaa=Homoleucine
KKKKKKKKRPRXYXX

SEQ ID NO: 33 - protein, 17 aa, Artificial
  Synthetic peptide
  MISC_FEATURE (1)..(1): DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (7)..(7): DLys
  MISC_FEATURE (8)..(8): DLys
  MISC_FEATURE (9)..(9): DLys
  MISC_FEATURE (10)..(10): DLys
  MISC_FEATURE (14)..(14): Xaa=Nva
  MISC_FEATURE (16)..(16): Xaa=Dap
  MISC_FEATURE (17)..(17): Xaa=Homoleucine
KKKKKKKKKKRPRXYXX

SEQ ID NO: 34 - protein, 8 aa, Artificial
  Synthetic peptide
  MISC_FEATURE (4)..(4): Xaa=Nva
  MISC_FEATURE (6)..(6): Xaa=Dap
  MISC_FEATURE (7)..(7): Xaa=Homoleucine
  MISC_FEATURE (8)..(8): Xaa=(DArg)7
RPRXYXXX

SEQ ID NO: 35 - protein, 18 aa, Artificial
  Synthetic peptide
  MISC_FEATURE (1)..(1): Xaa=Ahx6 (6-amino hexanoic acid)
  MISC_FEATURE (2)..(2): DArg
  MISC_FEATURE (3)..(3): DArg
  MISC_FEATURE (4)..(4): DArg
  MISC_FEATURE (5)..(5): DArg
  MISC_FEATURE (6)..(6): DGln
  MISC_FEATURE (7)..(7): DArg
  MISC_FEATURE (8)..(8): DArg
  MISC_FEATURE (9)..(9): DLys
  MISC_FEATURE (10)..(10): DLys
  MISC_FEATURE (11)..(11): DArg
  MISC_FEATURE (15)..(15): Xaa=Nva
  MISC_FEATURE (17)..(17): Xaa=Dap
  MISC_FEATURE (18)..(18): Xaa=Homoleucine
XRRRRQRRKKRRPRXYXX

SEQ ID NO: 36 - protein, 8 aa, Artificial
  Synthetic peptide
  MISC_FEATURE (1)..(1): Xaa=(DArg)7
  MISC_FEATURE (5)..(5): Xaa=Nva
  MISC_FEATURE (7)..(7): Xaa=Dap
  MISC_FEATURE (8)..(8): Xaa=Homoleucine
XRPRXYXX

SEQ ID NO: 37 - protein, 7 aa, Artificial
  Synthetic peptide
  MISC_FEATURE (1)..(1): Xaa=Biotinyl-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 38 - protein, 7 aa, Artificial
  Synthetic peptide
  MISC_FEATURE (1)..(1): Xaa=Laoryl-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 39 - protein, 7 aa, Artificial
  Synthetic peptide
  MISC_FEATURE (1)..(1): Xaa=S-carbofluorescin-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 40 - protein, 8 aa, Artificial
  Synthetic peptide
  MISC_FEATURE (1)..(1): Xaa=Lithocholyl-Gly
  MISC_FEATURE (5)..(5): Xaa=Nva
  MISC_FEATURE (8)..(8): Xaa=Homoleucine
XRPRXYAX

SEQ ID NO: 41 - protein, 7 aa, Artificial
  Synthetic peptide
  MISC_FEATURE (1)..(1): Xaa=Lithocholyl-Arg
  MISC_FEATURE (6)..(6): Xaa=Nva
  MISC_FEATURE (7)..(7): Xaa=Homoleucine
XPRXYAX

SEQ ID NO: 42 - protein, 7 aa, Artificial
  Synthetic peptide
  MISC_FEATURE (1)..(1): Xaa=H-1-adamantyl-Arg
  MISC_FEATURE (4)..(4): Xaa=Nva
  MISC_FEATURE (7)..(7): Xaa=Homoleucine
XPRXYAX

SEQ ID NO: 43 - protein, 7 aa, Artificial
  Synthetic peptide
  MISC_FEATURE (1)..(1): Xaa=1-adamantyl-CH2-CO-Arg
  MISC_FEATURE (4)..(4): Xaa=Nva
  MISC_FEATURE (7)..(7): Xaa=Homoleucine
XPRXYAX

SEQ ID NO: 44 - protein, 7 aa, Artificial
  Synthetic peptide
  MISC_FEATURE (1)..(1): Xaa=Testosterone-yl-O-CO-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 45 - protein, 7 aa, Artificial
  Synthetic peptide
  MISC_FEATURE (1)..(1): Xaa=Galactopyranosyl-O-CO-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 46 - protein, 8 aa, Artificial
  Synthetic peptide
  MISC_FEATURE (1)..(1): Xaa=Lithocholyl-Gly
  MISC_FEATURE (5)..(5): Xaa=Nva
  MISC_FEATURE (7)..(7): Xaa=Dap
  MISC_FEATURE (8)..(8): Xaa=Homoleucine
XRPRXYXX

SEQ ID NO: 47 - protein, 7 aa, Artificial
  Synthetic peptide
  MISC_FEATURE (1)..(1): Xaa=Lithocholyl-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 48 - protein, 7 aa, Artificial
  Synthetic peptide
  MISC_FEATURE (1)..(1): Xaa=Galactopyranosyl-O-CO-Arg
  MISC_FEATURE (4)..(4): Xaa=Nva
  MISC_FEATURE (7)..(7): Xaa=Homoleucine
XPRXYAX

SEQ ID NO: 49 - protein, 7 aa, Artificial
  Synthetic peptide
  MISC_FEATURE (1)..(1): Xaa=Testosterone-yl-O-CO-Arg
  MISC_FEATURE (4)..(4): Xaa=Nva
  MISC_FEATURE (7)..(7): Xaa=Homoleucine
XPRXYAX

SEQ ID NO: 50 - protein, 7 aa, Artificial
  Synthetic peptide
  MISC_FEATURE (1)..(1): Xaa=VitB6-Arg
  MISC_FEATURE (4)..(4): Xaa=Nva
  MISC_FEATURE (7)..(7): Xaa=Homoleucine
XPRXYAX

SEQ ID NO: 51 - protein, 7 aa, Artificial
  Synthetic peptide
  MISC_FEATURE (1)..(1): Xaa=VitB6-Arg
  MISC_FEATURE (4)..(4): Xaa=Nva
  MISC_FEATURE (6)..(6): Xaa=Dap
  MISC_FEATURE (7)..(7): Xaa=Homoleucine
XPRXYXX

SEQ ID NO: 52 - protein, 7 aa, Artificial
  Synthetic peptide
  MISC_FEATURE (1)..(1): Xaa=VitE-Succinate-Arg
  MISC_FEATURE (4)..(4): Xaa=Nva
  MISC_FEATURE (7)..(7): Xaa=Homoleucine
XPRXYAX

SEQ ID NO: 53 - protein, 15 aa, Artificial
  Synthetic peptide
  MISC_FEATURE (1)..(1): Xaa=Cholesteryl-DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (7)..(7): DLys
  MISC_FEATURE (8)..(8): DLys
  MISC_FEATURE (12)..(12): Xaa=Nva
  MISC_FEATURE (14)..(14): Xaa=Dap
  MISC_FEATURE (15)..(15): Xaa=Homoleucine
  MOD_RES (15)..(15): AMIDATION
XKKKKKKKRPRXYXX

SEQ ID NO: 54 - protein, 8 aa, Artificial
  Synthetic peptide
  MISC_FEATURE (1)..(1): Xaa=Cholesteryl-CO-Gly
  MISC_FEATURE (5)..(5): Xaa=Nva
  MISC_FEATURE (7)..(7): Xaa=Dap
  MISC_FEATURE (8)..(8): Xaa=Homoleucine
  MOD_RES (8)..(8): AMIDATION
XRPRXYXX

SEQ ID NO: 55 - protein, 8 aa, Artificial
  Synthetic peptide
  MISC_FEATURE (1)..(1): Xaa=Cholesteryl-O-CO-betaAla
  MISC_FEATURE (5)..(5): Xaa=Nva
  MISC_FEATURE (7)..(7): Xaa=Dap
  MISC_FEATURE (8)..(8): Xaa=Homoleucine
  MOD_RES (8)..(8): AMIDATION
XRPRXYXX

SEQ ID NO: 56 - protein, 8 aa, Artificial
  Synthetic peptide
  MISC_FEATURE (1)..(1): Xaa=Cholesteryl-O-CO-GABA
  MISC_FEATURE (5)..(5): Xaa=Nva
  MISC_FEATURE (7)..(7): Xaa=Dap
  MISC_FEATURE (8)..(8): Xaa=Homoleucine
  MOD_RES (8)..(8): AMIDATION
XRPRXYXX

SEQ ID NO: 57 - protein, 7 aa, Artificial
  Synthetic peptide
  MISC_FEATURE (1)..(1): Xaa=Cholesteryl-O-CO-Arg
  MISC_FEATURE (4)..(4): Xaa=Orn
  MISC_FEATURE (5)..(5): Xaa=Glu(aminopropyl mercaptoisoquinoline)
  MOD_RES (7)..(7): AMIDATION
XPRXXSF

SEQ ID NO: 58 - protein, 7 aa, Artificial
  Synthetic peptide
  MISC_FEATURE (1)..(1): Xaa=Cholesteryl-O-CO-Arg
  MISC_FEATURE (4)..(4): Xaa=Nva
  MISC_FEATURE (6)..(6): Xaa=Ser(Me)
  MISC_FEATURE (7)..(7): Xaa=Homoleucine
  MOD_RES (7)..(7): AMIDATION
XPRXYXX

SEQ ID NO: 59 - protein, 8 aa, Artificial
  Synthetic peptide
  MISC_FEATURE (1)..(1): Xaa=Cholesteryl-O-CO-Gly
  MISC_FEATURE (5)..(5): Xaa=Nva
  MISC_FEATURE (7)..(7): Xaa=Ser(Me)
  MISC_FEATURE (8)..(8): Xaa=Homoleucine
  MOD_RES (8)..(8): AMIDATION
XRPRXYXX

SEQ ID NO: 60 - protein, 8 aa, Artificial
  Synthetic peptide
  MISC_FEATURE (1)..(1): Xaa=Cholesteryl-O-CO-betaAla
  MISC_FEATURE (5)..(5): Xaa=Nva
  MISC_FEATURE (7)..(7): Xaa=Ser(Me)
  MISC_FEATURE (8)..(8): Xaa=Homoleucine
  MOD_RES (8)..(8): AMIDATION
XRPRXYXX

SEQ ID NO: 61 - protein, 8 aa, Artificial
  Synthetic peptide
  MISC_FEATURE (1)..(1): Xaa=Cholesteryl-O-CO-GABA
  MISC_FEATURE (5)..(5): Xaa=Nav
  MISC_FEATURE (7)..(7): Xaa=Ser(Me)
  MISC_FEATURE (8)..(8): Xaa=Homoleucine
  MOD_RES (8)..(8): AMIDATION
XRPRXYXX

SEQ ID NO: 62 - protein, 16 aa, Artificial
  Synthetic peptide
  MISC_FEATURE (1)..(1): DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (7)..(7): DLys
  MISC_FEATURE (8)..(8): DLys
  MISC_FEATURE (12)..(12): Xaa=Nva
  MISC_FEATURE (14)..(14): Xaa=Dap
  MISC_FEATURE (15)..(15): Xaa=Homoleucine
  MISC_FEATURE (16)..(16): Xaa=Lys(CO-cholesteryl)
  MOD_RES (16)..(16): AMIDATION
KKKKKKKKRPRXYXXX

SEQ ID NO: 63 - protein, 24 aa, Artificial
  Synthetic peptide
  MISC_FEATURE (20)..(20): Xaa=Nva
  MISC_FEATURE (22)..(22): Xaa=Dap
  MISC_FEATURE (23)..(23): Xaa=Homoleucine
  MISC_FEATURE (24)..(24): Xaa=Lys(CO-cholesteryl)
  MOD_RES (24)..(24): AMIDATION
RQIKIWFQNRRMKWKKRPRXYXXK

SEQ ID NO: 64 - protein, 12 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (9)..(9): Xaa=Nva
  MISC_FEATURE (11)..(11): Xaa=Dap
  MISC_FEATURE (12)..(12): Xaa=Homoleucine
  MOD_RES (12)..(12): AMIDATION
XKKKKRPRXYXX

SEQ ID NO: 65 - protein, 13 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (4)..(4): DLys
  MISC_FEATURE (5)..(5): DLys
  MISC_FEATURE (6)..(6): DLys
  MISC_FEATURE (10)..(10): Xaa=Nva
  MISC_FEATURE (12)..(12): Xaa=Dap
  MISC_FEATURE (13)..(13): Xaa=Homoleucine
  MOD_RES (13)..(13): AMIDATION
XKKKKKRPRXYXX

SEQ ID NO: 66 - protein, 9 aa, Artificial
  Synthetic peptide
  MISC_FEATURE (1)..(1): Xaa=Cholesteryl-O-CO-Gly
  MISC_FEATURE (6)..(6): Xaa=Nva
  MISC_FEATURE (8)..(8): Xaa=Dap
  MISC_FEATURE (9)..(9): Xaa=Homoleucine
  MOD_RES (9)..(9): AMIDATION
XGRPRXYXX

SEQ ID NO: 67 - protein, 10 aa, Artificial
  Synthetic peptide
  MISC_FEATURE (1)..(1): Xaa=Cholesteryl-O-CO-Gly
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XGGRPRXYXX

SEQ ID NO: 68 - protein, 11 aa, Artificial
  Synthetic peptide
  MISC_FEATURE (1)..(1): Xaa=Cholesteryl-O-CO-Gly
  MISC_FEATURE (8)..(8): Xaa=Nva
  MISC_FEATURE (10)..(10): Xaa=Dap
  MISC_FEATURE (11)..(11): Xaa=Homoleucine
  MOD_RES (11)..(11): AMIDATION
XGGGRPRXYXX

SEQ ID NO: 69 - protein, 12 aa, Artificial
  Synthetic peptide
  MISC_FEATURE (1)..(1): Xaa=Cholesteryl-O-CO-Gly
  MISC_FEATURE (9)..(9): Xaa=Nva
  MISC_FEATURE (11)..(11): Xaa=Dap
  MISC_FEATURE (12)..(12): Xaa=Homoleucine
  MOD_RES (12)..(12): AMIDATION
XGGGGRPRXYXX

SEQ ID NO: 70 - protein, 13 aa, Artificial
  Synthetic peptide
  MISC_FEATURE (1)..(1): Xaa=Cholesteryl-O-CO-Gly
  MISC_FEATURE (10)..(10): Xaa=Nva
  MISC_FEATURE (12)..(12): Xaa=Dap
  MISC_FEATURE (13)..(13): Xaa=Homoleucine
  MOD_RES (13)..(13): AMIDATION
XGGGGGRPRXYXX

SEQ ID NO: 71 - protein, 7 aa, Artificial
  Synthetic peptide
  MISC_FEATURE (1)..(1): Xaa=Decanyl-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 72 - protein, 7 aa, Artificial
  Synthetic peptide
  MISC_FEATURE (1)..(1): Xaa=Cholesteryl-O-CO-(CH2)2-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 73 - protein, 7 aa, Artificial
  Synthetic peptide
  MISC_FEATURE (1)..(1): Xaa=Sperminyl-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 74 - protein, 7 aa, Artificial
  Synthetic peptide
  MISC_FEATURE (1)..(1): Xaa=Z-2-AcO-cinnamoyl-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 75 - protein, 10 aa, Artificial
  Synthetic peptide
  MISC_FEATURE (1)..(1): Xaa=Z-2-AcO-cinnamoyl-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 76 - protein, 7 aa, Artificial
  Synthetic peptide
  MISC_FEATURE (1)..(1): Xaa=NH2-(CH2)3-NH-(CH2)3-NH-CH2-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 77 - protein, 7 aa, Artificial
  Synthetic peptide
  MISC_FEATURE (1)..(1): Xaa=H-His
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 78 - protein, 7 aa, Artificial
  Synthetic peptide
  MISC_FEATURE (1)..(1): Xaa=H-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPHXYXX

SEQ ID NO: 79 - protein, 7 aa, Artificial
  Synthetic peptide
  MISC_FEATURE (1)..(1): Xaa=H-His
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPHXYXX

SEQ ID NO: 80 - protein, 7 aa, Artificial
  Synthetic peptide
  MISC_FEATURE (1)..(1): Xaa=H-Arg
  MISC_FEATURE (3)..(3): Xaa=Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPXXYXX

SEQ ID NO: 81 - protein, 7 aa, Artificial
  Synthetic peptide
  MISC_FEATURE (1)..(1): Xaa=Cholesteryl-O-CO-His
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 82 - protein, 7 aa, Artificial
  Synthetic peptide
  MISC_FEATURE (1)..(1): Xaa=Cholesteryl-O-CO-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPHXYXX

SEQ ID NO: 83 - protein, 7 aa, Artificial
  Synthetic peptide
  MISC_FEATURE (1)..(1): Xaa=Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 84 - protein, 7 aa, Artificial
  Synthetic peptide
  MISC_FEATURE (1)..(1): Xaa=Orn
  MISC_FEATURE (3)..(3): Xaa=Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPXXYXX

SEQ ID NO: 85 - protein, 7 aa, Artificial
  Synthetic peptide
  MISC_FEATURE (1)..(1): Xaa=Cholesteryl-O-CO-Orn
  MISC_FEATURE (3)..(3): Xaa=Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPXXYXX

SEQ ID NO: 86 - protein, 7 aa, Artificial
  Synthetic peptide
  MISC_FEATURE (1)..(1): Xaa=Cholesteryl-O-CO-Arg
  MISC_FEATURE (3)..(3): Xaa=Orn
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPXXYXX

SEQ ID NO: 87 - protein, 6 aa, Artificial
  Synthetic peptide
  MISC_FEATURE (1)..(1): Xaa=Cholesteryl-Arg
  MISC_FEATURE (4)..(4): Xaa=Nva
  MISC_FEATURE (6)..(6): Xaa=Dap-OH
XPRXYX

SEQ ID NO: 88 - protein, 7 aa, Artificial
  Synthetic peptide
  MISC_FEATURE (1)..(1): Xaa=Coumarin-3-oyl-Arg
  MISC_FEATURE (4)..(4): Xaa=Nva
  MISC_FEATURE (6)..(6): Xaa=Dap
  MISC_FEATURE (7)..(7): Xaa=Homoleucine
  MOD_RES (7)..(7): AMIDATION
XPRXYXX

SEQ ID NO: 89 - protein, 10 aa, Artificial
  Synthetic peptide
  MISC_FEATURE (1)..(1): Xaa=HO-CO-(CH2)2-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKRPRXYXX

SEQ ID NO: 90 - protein, 10 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (4)..(4): Xaa=Orn
  MISC_FEATURE (7)..(7): Xaa=Nva
  MISC_FEATURE (9)..(9): Xaa=Dap
  MISC_FEATURE (10)..(10): Xaa=Homoleucine
  MOD_RES (10)..(10): AMIDATION
XKKXPRXYXX

SEQ ID NO: 91 - protein, 11 aa, Artificial
  Synthetic peptide
  MISC_FEATURE (1)..(1): Xaa=Cholesteryl-O-CO-DLys
  MISC_FEATURE (2)..(2): DLys
  MISC_FEATURE (3)..(3): DLys
  MISC_FEATURE (7)..(7): Xaa=Nva
  MISC_FEATURE (9)..(9): Xaa=Dap
  MISC_FEATURE (10)..(10): Xaa=Homoleucine
  MISC_FEATURE (11)..(11): Xaa=Lys(FITC)
  MOD_RES (11)..(11): AMIDATION
XKKRPRXYXXX

SEQ ID NO: 92 - protein, 16 aa, Homo sapiens
RQIKIWFQNRRMKWKK

SEQ ID NO: 93 - protein, 10 aa, Artificial
  Synthetic peptide
  MISC_FEATURE (1)..(1): Xaa=Ahx6 (6-amino hexanoic acid)
  MISC_FEATURE (2)..(2): DArg
  MISC_FEATURE (3)..(3): DArg
  MISC_FEATURE (4)..(4): DArg
  MISC_FEATURE (5)..(5): DArg
  MISC_FEATURE (6)..(6): DGln
  MISC_FEATURE (8)..(8): DLys
  MISC_FEATURE (9)..(9): DLys
  MISC_FEATURE (10)..(10): DArg
XRRRRQRKKR

(19) United States
(12) Patent Application Publication
(10) Pub. No.: US 2007/0078092 A1
(43) Pub. Date: Apr. 5, 2007

(54) Cell permeable conjugates of peptides for inhibition of protein kinases

(76) Inventors: Nurit Livnah, Mazkeret Batya (IL); Alexander Levitzki, Jerusalem (IL); Hanoch Senderovitz, Tel Aviv (IL); Tamar Yechezkel, Ramat-Gan (IL); Yosef Salitra, Rehovot (IL); Pninit Litman, Ziona (IL); Osnat Ohne, Tel Mond (IL)

Correspondence Address: WINSTON & STRAWN LLP;PATENT DEPARTMENT, 1700 K STREET, N.W., WASHINGTON, DC, 20006, US

(21) Appl. No.: 11/295,793

(22) Filed: Dec. 6, 2005

Related U.S. Application Data
(63) Continuation of application No. PCT/IL04/00505, filed on Jun. 13, 2004.

(30) Foreign Application Priority Data
Jun. 12, 2003 (IL) IL 156429

(51) Int. Cl. A61K 38/55; A61K 38/08; C12N 9/99

(52) U.S. Cl. 514/16; 530/329; 435/184

(57) ABSTRACT

The present invention provides inhibitors of protein kinases comprising a molecule having at least a first moiety competent for penetration of the molecule into cells, and a second moiety for having a protein kinase inhibiting effect within the cells. The first moiety is joined to the second moiety through a linker or a spacer. The complex molecules are preferably peptide conjugates having improved cell-permeability, serum stability and kinase selectivity compared to known protein kinase inhibitors. Pharmaceutical compositions that include these protein kinase inhibitors, and methods of using such compositions for treatment of cancers and other diseases associated with protein kinase activity are also disclosed.

### CROSS-REFERENCE TO RELATED APPLICATIONS

This application is a continuation of International application PCT/IL2004/000505 filed Jun. 13, 2004, the entire content of which is expressly incorporated herein by reference thereto.

### FIELD OF THE INVENTION

The present invention relates to cell permeable, stable conjugates comprising a cell-permeability enhancement moiety and a peptide or peptidomimetic, as selective inhibitors of protein kinases, to pharmaceutical compositions containing them, as well as to processes for the preparation and use of such complex molecules.

### BACKGROUND OF THE INVENTION

Protein kinases are involved in signal transduction pathways linking growth factors, hormones and other cell regulation molecules to cell growth, survival and metabolism under both normal and pathological conditions. The superfamily of protein kinases includes protein kinase A and protein kinase C, as well as the more recently discovered protein kinase B (PKB).

PKB is an anti-apoptotic protein kinase whose activity is strongly elevated in human malignancies. PKB was originally discovered as a viral oncogene v-Akt in rat T-cell leukemia. It was later established that v-Akt is the oncogenic version of a cellular enzyme PKB/c-Akt, in which a truncated viral group specific antigen, gag, is fused in frame to the full length Akt−1 and is membrane bound whereas PKB/c-Akt is cytoplasmic. Sequencing of Akt revealed a high degree of homology to PKA (˜75%) and PKC isozymes (˜50%), a fact which led to its renaming as PKB.

PKB activation involves phosphorylation of two amino acid residues, Ser473 and Thr308. The enzyme is activated by the second messenger PIP3 produced by PI′-3-kinase. PIP3 binds to the pleckstrin homology (PH) domains of PKB, recruits it to the membrane where it is phosphorylated and converted to its activated form. Since PKB activation is PI′-3-kinase dependent, the persistent activation of certain protein tyrosine kinases, such as IGF−1 receptor, EGF receptor, PDGF receptor, pp60c-Src, and the like, leads to the persistent activation of PKB which is indeed encountered in many tumors. Deletions in the gene coding for the tumor suppressor PTEN also induce the persistent activation of PKB/cAkt since it is the negative regulator of this enzyme. Also, PKB is overexpressed in 15% of ovarian cancers, 12% of pancreatic cancers and 3% of breast cancers, and was shown to produce a survival signal that protects cells from apoptosis thus contributing to resistance to chemotherapy.

PKB has emerged as a crucial regulator of widely divergent cellular processes including apoptosis, proliferation, differentiation and metabolism. Disruption of normal PKB/Akt signaling has now been documented as a frequent occurrence in several human cancers and the enzyme appears to play an important role in their progression (Nicholson and Anderson, Cellular Signalling 14, 381, 2002).

These molecular properties of PKB and its central role in tumorigenesis, implies that this protein kinase may be an attractive target for novel anti-cancer agents. Ideally, a drug that inhibits PKB should cause both cell cycle arrest and promote apoptosis. Such activity would result in increased cell death of tumor tissue where PKB is elevated, and in decreased resistance to chemotherapy agents.

Prostate cancer is the most frequently diagnosed cancer in men and is responsible for approximately 41,000 deaths in the United States annually (Parker, S. L., et al., 1996, CA Cancer J. Clin., 65:5-27). Early stage, organ-confined, prostate cancer is often managed with surgery or radiation therapy until the patient dies from unrelated causes.

Hormone-Refractory-Prostate Cancer (HRPC), a non curable cancer type, is the second leading cause of cancer deaths in men in the US. Direct correlation between resistance to chemotherapy and activation of PKB was shown in several prostate cancer cell lines and in human tumorogenic tissues, and elevation of PKB levels in prostate tumor tissues, is clinically associated with HRPC (Yongde et al., 2003, Int.J.Cancer: 107, 676-680). High correlation of PKB levels with the Gleason pattern and PSA (Prostate specific Antigen) levels in prostate cancer patients indicates the significant role of PKB in this type of cancer.

Hill M M, and Hemmings B A, (Pharmacol Ther. 2002, 93, 243-51,2002) describe the involvement of PBK in other tumors such as ovarian, breast and pancreatic.

A potent protein kinase inhibitor has to include a substrate mimetic which is usually based on the peptidic stricture of the substrate, and/or an ATP mimetic.

Hidaka H. et al. (Biochemistry, 32, 5036, 1984) describe a class of isoquinolinesulfonamides having inhibitory activity towards cyclic nucleotide dependent protein kinases (PKA and PKG) and protein kinases C (PKC). Additional derivatives of isoquinolinesulfonyl were disclosed by Hidaka in EP 109023, U.S. Pat. No. 4456757, U.S. Pat. No. 4525589, and U.S. Pat. No. 4560755.

International PCT application WO 93/13072 discloses 5-isoquinolinesulfonamide derivatives as protein kinase inhibiting agents. International PCT application WO 98/53050 discloses short peptides derived from the HJ loop of a serine/threonine kinase which modulate the activity of serine/threonine kinases.

The minimal consensus sequence for efficient phosphorylation by PKB was found by Alessi et al. (Fed. Eur. Biochem. Soc., 399, 333, 1996). This is a 7-mer motif with the most active peptide substrate having the sequence Arg-Pro-Arg-Thr-Ser-Ser-Phe (SEQ ID NO:1). International application WO 97/22360 discloses certain PKB substrate peptides having 7-amino acids length, useful as substrate for measuring PKB activity.

Obata et al. (J. Biol. Chem., 17, 36108, 2000) described the use of an oriented peptide library approach to determine optimal amino acid sequence of the PKB substrate. All the substrates identified contained the known motif having the sequence Arg-Xaa-Arg-Xaa-Saa-Ser/Thr.

Ricouart et al. (J. Med. Chem. 1991, 34, 73-78), described conjugates of isoquinolinesulfonamides and peptides for the inhibition of PKA. Loog et al. (Bioorganic and Medicinal Chemistry Letters 1999, 9, 1447-1452), described a chimera with adenosine and peptides for the inhibition of PKA and PKC. The inhibition obtained with the disclosed compound is very poor. Schlitzer et al. (Bioorganic and Medicinal Chemistry, 2000, 1991-2006) deal with a small molecule linked to non-peptidic long chain moieties that are intended to replace the peptide part of the substrate. The disclosed compounds show poor inhibitory activity.

Parang et al. (Nature Structural Biology 8, 37, 2001), describe peptide-ATP bisubstrate analogs of a protein kinase A inhibitor, wherein ATP is linked to a protein kinase peptide substrate. Nevertheless, this approach has a limitation of suboptimal pharmacokinetic properties. WO 01/70770 discloses bisubstrate inhibitors for the insulin receptor tyrosine kinase, and a specific potent and selective inhibitor comprising an ATP-gamma-S linked to a peptide substrate analog via a two-carbon spacer.

International Patent Application WO 01/91754 by one of the present inventors and colleagues relates to specific isoquinoline derivatives, which are PKB inhibitors.

The applicants of the present invention disclose in WO 03/010281 bi-substrate protein kinase inhibitors comprising ATP mimetics conjugated to peptides or peptidomimetics. Small molecules (particularly isoquinoline derivatives) having high affinity to the ATP binding site of protein kinases, are conjugated to a peptide or peptidomimetic which mimics the substrate of PKB. Some of the peptides themselves were highly active and selective but were not stable in serum and not active in cells and therefore have low therapeutic value. These chimeric compounds demonstrate increased activity but decreased selectivity in comparison to the peptides, due to the presence of the ATP mimetic moiety. Furthermore, the chimeric compounds showed low activity in cells, and low to moderate stability in serum.

Lindgren et al. (TIPS 21, 99-103 2000) reviews cellular delivery using cell penetrating peptides.

Chemotherapy and Combination Therapy

Combatting the growth of neoplastic cells and tumors has been a major focus of biological and medical research. Such research has led to the discovery of novel cytotoxic agents potentially useful in the treatment of neoplastic disease. Examples of cytotoxic agents commonly employed in chemotherapy include anti-metabolic agents interfering with microtubule formation, alkylating agents, platinum-based agents, anthracyclines, antibiotic agents, topoisomerase inhibitors, and other agents.

Aside from merely identifying potential chemotherapeutic agents, cancer research has led to an increased understanding of the mechanisms by which these agents act upon neoplastic cells, as well as on other cells. For example, cholecalciferol (vitamin D) can effect differentiation and reduce proliferation of several cell types cells both in vitro and in vivo. The active metabolite of vitamin D and analogs mediate significant in vitro and in vivo anti-tumor activity by retarding the growth of established tumors and preventing tumor induction (Colston et al. 1989, Lancet, 1, 188; Belleli et al. 1992, Carcinogenesis, 13, 2293; McElwain et al. 1995, Mol. Cell. Diff., 3, 31-50; Clark et al. 1992, J. Cancer Res. Clin. Oncol., 118, 190; Zhou et al. 1989, Blood, 74, 82-93).

Platinum-based agents are widely utilized in chemotherapeutic applications. For example, cisplatin kills tumor cells via formation of covalent, cross- or intrastrand DNA adducts (Sherman et al. 1987, Chem. Rev., 87, 1153-81; Chu, J. 1994, Biol. Chem., 269, 787-90). Treatment with such platinum-based agents thereby leads to the inhibition of DNA synthesis (Howle et al. 1970, Biochem. Pharmacol., 19, 2757-62; Salles et al. 1983, Biochem. Biophys. Res. Commun., 112, 555-63).

Other chemotherapeutic agents act by different mechanisms. For example, agents interfering with microtubule formation (e.g., vincristine, vinblastine, paclitaxel, docetaxel, etc.) act against neoplastic cells by interfering with proper formation of the mitotic spindle apparatus (see, e.g., Manfredi et al. 1984, Pharmacol. Ther., 25, 83-125). Thus, agents interfering with microtubule formation mainly act during the mitotic phase of the cell cycle (Schiff et al. 1980, Proc. Nat. Acad. Sci. U.S.A., 77, 1561-65; Fuchs et al. 1978, Cancer Treat. Rep., 62, 1219-22; Lopes et al. 1993, Cancer Chemother. Pharmacol., 32, 235-42). Antimetabolites act on various enzymatic pathways in growing cells. For example, methotrexate (MTX) is a folic acid analog which inhibits dihydrofolate reductase. As a result, it blocks the synthesis of thymidylate and purines required for DNA synthesis. Other cytotoxic agents can also be employed (e.g., taxanes such as docetaxel (e.g., TAXATERE®).

One of the major problems in cancer therapy today is the ability of tumor cells to develop resistance to chemotherapeutic agents. Because of the differences in the biological mechanisms of various cytotoxic agents, protocols involving combinations of different cytotoxic agents have been attempted (e.g., Jekunen et al. 1994, Br. J. Cancer, 69, 299-306; Yeh et al. 1994, Life Sciences, 54, 431-35). Combination treatment protocols aim to increase the efficacy of cytopathic protocols by using compatible cytotoxic agents. In turn, the possibility that sufficient antineoplastic activity can be achieved from a given combination of cytotoxic agents presents the possibility of reducing the dosage of individual cytotoxic agents to minimize harmful side effects. In part because the various cytotoxic agents act during different phases of the cell cycle, the success of combination protocols frequently depends upon the order of drug application (e.g., Jekunen et al., supra; Studzinski et al. 1991, Cancer Res., 51, 3451).

U.S. Pat. No. 6,559,139 describes combination therapy using vitamin D or derivatives thereof with other known chemotherapy agent. U.S. Pat. No. 6,667,337 discloses method of treating cancer using combination of a compound of the xanthenone acetic acid class and either paclitaxel or docetaxel.

U.S. Pat. No. 5,985,877 discloses combination of tyrosine kinase inhibitor and chemical castration to treat prostate cancer.

U.S. Pat. Nos. 5,516,771, 5,654,427 and 5,650,407 discuss indolocarbazole-type tyrosine kinase inhibitors and prostate cancer. U.S. Pat. Nos. 5,475,110; 5,591,855; and 5,594,009; and WO 96/11933 discuss fused pyrrolocarbazole-type tyrosine kinase inhibitors and prostate cancer.

There continues to be a need for effective treatment regimens in a variety of cancers, including prostate cancer. The present invention overcomes the limitations of known anti proliferation and anti cancer agents by providing inhibitors of protein kinases, comprising peptide and peptidomimetic conjugates having improved pharmacological properties such as cell permeability, selectivity and resistance to biodegradation.

### SUMMARY OF THE INVENTION

The present invention provides novel protein kinase inhibitors comprising conjugates of a peptide or a peptidomimetic with a cell permeability enhancer.

The present invention fulfills the unmet need for a specific inhibitor of protein kinase B which is able to cause both cell cycle arrest and promote apoptosis leading to increased cell death of tumor tissue where PKB is elevated, and in decreased resistance to known chemotherapy agents. The combination of the protein kinase inhibitors of the present invention with other therapeutics provide enhanced clinical efficacy and/or a reduced occurrence of adverse side effects which would allow for administration of higher doses of conventional chemotherapeutics.

The present invention further provides a protein kinase inhibitor, comprising a molecule having at least a first moiety competent for penetration of the molecule into cells, and a second moiety for having a protein kinase inhibiting effect within the cells, the first moiety being joined to the second moiety through a linker or spacer. In particular the present invention provides cell permeable peptide and peptidomimetic conjugates that are inhibitors of protein kinases for medical and therapeutic purposes.

According to certain embodiments the conjugates of the present invention comprise a peptide substrate mimetic linked to a cell-permeability moiety.

According to additional embodiments the conjugates of the present invention comprise peptides and peptidomimetics that are selective inhibitors of protein kinase B (PKB). These peptide and peptidomimetic conjugates have improved pharmacological properties over previously described PKB inhibitors.

Accordingly, the peptide conjugates of the present invention comprise a first segment of a cell penetration moiety and a second segment of a peptide or peptidomimetic -which serves as a peptidic core. The first segment and the second part may be linked directly via a covalent bond or through a spacer.

Any moiety known in the art to actively or passively facilitate or enhance permeability of the compound into cells may be used for conjugation with the peptide core according to the present invention. Non-limitative examples include: hydrophobic moieties such as fatty acids, steroids and bulky aromatic or aliphatic compounds; moieties which may have cell-membrane receptors or carriers, such as steroids, vitamins and sugars, natural and non-natural amino acids and transporter peptides.

The protein kinase inhibitory peptide moiety of the complex molecules according to the present invention is selected from a protein kinase inhibitory peptide or a protein kinase inhibitory peptidomimetic. Such inhibitory core peptides are designed based on known or novel peptides and peptidomimetics which constitute a PKB substrate or a PKB substrate mimetic. The peptidic core according to the present invention generally comprises a sequence of 4-25 amino acids, according to certain embodiments the peptidic core comprises 5-20 amino acids, while according to yet another embodiment it comprises 6-15 amino acids.

According to a specific embodiment of the present invention, the peptide moiety is derived from a PKB substrate. According to a more preferred embodiment, the peptidic core is derived from the PKB substrate glycogen synthase kinase 3 (GSK3).

According to another embodiment the conjugates of the invention may further comprise an ATP mimetic moiety.

The cell-permeability moiety may be connected to any position in the peptide moiety, directly or through a spacer. According to specific embodiments, the cell-permeability moiety is connected to the amino or carboxy terminus of the peptide moiety. The optional connective spacer may be of varied lengths and conformations comprising any suitable chemistry including but not limited to amine, amide, carbamate, thioether, oxyether, sulfonamide bond and the like. Non-limiting examples for such spacers include amino acids, sulfone amide derivatives, amino thiol derivatives and amino alcohol derivatives.

The present invention also provides peptide-based protein kinase inhibitors comprising peptidomimetic compounds having further improved stability and cell permeability properties. Non limiting examples of such compounds include N-alkylation of selected peptide residues, side-chain modifications of selected peptide residues, non-natural amino acids, use of carbamate, urea, sulfonamide and hydrazine for peptide bond replacement, and incorporation of non-peptide moieties including but not limited to piperidine, piperazine and pyrrolidine, through a peptide or non-peptide bond. Modified bonds between amino acids (AAs) in peptidomimetic cores according to the present invention may be selected from the group consisting of: an amide, urea, carbamate, hydrazine or sulfonamide bond. In the currently more preferred embodiments the bonds between the AAs are all amide bonds unless explicitly stated otherwise.

The present invention further provides peptide-based, cell permeable chimeric compounds further comprising an ATP mimetic moiety. The ATP mimetic moiety includes but is not limited to dansyls, isoquinolines, quinolines and naphthalenes, and is optionally connected to the peptidic core by a spacer. Preferably, the ATP mimetic is an isoquinoline or its derivative. The spacer is of varied lengths and conformations of any suitable chemistry including but not limited to amine, amide, thioether, oxyether, sulfonamide bond and the like. Non-limiting examples for such spacers include sulfone amide derivatives, amino thiol derivatives and amino alcohol derivatives.

According to one embodiment compounds of the present invention comprise a sequence according to formula I:
M-X₁-Pro-Arg-X₄-X₅-X₆X₇ Formula I (SEQ ID NO:2)

wherein,

M is absent or is selected from D- or L-Lys₂₋₄;

X₁ is Arg, Lys, Orn or Dab;

X₄ is Nva, Leu, Ile, Abu or Orn;

X₅ is Tyr, Gly, GlyNH₂, Ser(Me), Glu, or Glu(NH—(CH2)2-NH—SO₂-isoquinoline);

X₆ is Dap, Abu, GlyNH2, Ser(Me), Gly, Ala or Ser; and

X₇ represents an aromatic or an aliphatic bulky residue, preferably Phe or Hol;

and analog, salt or functional derivative thereof.

According to specific embodiments a compound of the present invention comprises a sequence according to Formula II
M-Arg-Pro-Arg-X₄-X₅-X₆-X₇ Formula II (SEQ ID NO:3)

wherein,

M is DLys₃ or Lys₃;

X₄ is Nva, Leu, Ile, Abu or Orn;

X₅ is Tyr, Gly, GlyNH₂, Ser(Me), Glu, or Glu(NH—(CH2)2-NH—SO₂-isoquinoline);

X₆ is Dap, Abu, GlyNH2, Ser(Me), Gly, Ala or Ser; and

X₇ represents an aromatic or an aliphatic bulky residue, preferably Phe or Hol;

and analog, salt or functional derivative thereof.

According to another embodiment of the present invention the peptide conjugate comprises a cell-permeability moiety selected from the group consisting of: cholesterol, (DLys)₂₋₅, (Lys)₂₋₅, vitamin E, Arg-Gln-Ile-Lys-Ile-Trp-Phe-Gln-Asn-Arg-Arg-Met-Lys-Trp-Lys-Lys, Ahx6-DArg-DArg-DArg-DArg-DGln-Arg-DLys-DLys-DArg; (DLys)₈₋₁₀, and (DArg)₇₋₉.

According to specific embodiments of the present invention a protein kinase inhibitor comprises a sequence of Formula III:
Y-Z-Arg-Pro-Arg-Nva-Tyr-X₆-Hol Formula III (SEQ ID NO:4)
wherein X₆ is Dap or Ala; Y is a cell-permeability moiety; and Z is a spacer or a bond connecting Y to the peptide.

Preferably, Y is selected from the group consisting of: cholesterol, (DLys)₂₋₁₀, (Lys)₂₋₁₀, vitamin E, Arg-Gln-Ile-Lys-Ile-Trp-Phe-Gln-Asn-Arg-Arg-Met-Lys-Trp-Lys-Lys (SEQ ID NO :92), Ahx6-DArg-DArg-DArg-DArg-DGin-Arg-DLys-DLys-DArg (SEQ ID NO :93), and (DArg)₇₋₉; and Z absent or is selected from carbamate and Gly.

According to a further embodiment the protein kinase inhibitor comprises a conjugate comprising:

a. a peptide segment selected from the group consisting of:

DLys-DLys-DLys-Arg-Pro-Arg-Nva-Tyr-Dap-Hol (SEQ ID NO:5);

Lys-Lys-Lys-Arg-Pro-Arg-Nva-Tyr-Dap-Hol (SEQ ID NO:6);

Arg-Pro-Arg-Nva-Tyr-Dap-Hol (SEQ ID NO:7);

Arg-Pro-Arg-Orn-Glu-(NH-(CH2)2-NH—SO₂-Isoquinoline)Ser-Phe (SEQ ID NO:8); and

Arg-Pro-Arg-Nva-Tyr-Ala-Hol (SEQ ID NO:9); and

b. a cell-permeability moiety selected from the group of cholesterol, Arg-Gln-Ile-Lys-Ile -Trp-Phe-Gln-Asn-Arg-Arg-Met-Lys-Trp-Lys-Lys, vitamin E, and (DArg)₉.

Currently most preferred protein kinase inhibitors according to the present invention are selected from the group consisting of:
Cholesteryl-O—CO-DLys-DLys-DLys-Arg-Pro-Arg-Nva-Tyr-Dap-Hol-NH₂(SEQ ID NO:10);
Cholesteryl-O—CO-Lys-Lys-Lys-Arg-Pro-Arg-Nva-Tyr-Dap-Hol-NH₂(SEQ ID NO:11);
Cholesteryl-O—CO-(DLys)₄-Arg-Pro-Arg-Nva-Tyr-Dap-Hol-NH₂(SEQ ID NO:12);
Cholesteryl-O—CO-(DLys)₆-Arg-Pro-Arg-Nva-Tyr-Ser(Me)-Hol-NH₂(SEQ ID NO:13);
Cholesteryl-O—CO-Arg-Pro-Arg-Nva-Tyr-Dap-Hol-(DLys)₃-NH₂(SEQ ID NO:14);
Cholesteryl-O—CO-(DLys)₃-Arg-Pro-Arg-Nva-Tyr-Ser(Me)-Hol-NH2(SEQ ID NO:15);
Cholesteryl-O—CO-( Lys)₃-Arg-Pro-Arg-Nva-Tyr-Dap-Hol-OH (SEQ ID NO:16);
Cholesteryl-O—CO-(CH2)₂-CO-(DLys)₃-Arg-Pro-Arg-Nva-Tyr-Dap-Hol-NH₂(SEQ ID NO:17);
Cholesteryl-O—CO-Orn-Pro-Arg-Nva-Tyr-Dap-Hol-NH₂(SEQ ID NO:18);
Cholesteryl-O—CO-(DLys)₃-Lys-Pro-Arg-Nva-Tyr-Dap-Hol-NH₂(SEQ ID NO:19);
Vitamin E-CO—(CH2)₂-CO-DLys)₃-Arg-Pro-Arg-Nva-Tyr-Dap-Hol-NH2(SEQ ID NO:20);
Cholesteryl-O—CO-(DLyS)₂-Arg-Pro-Arg-Nva-Tyr-Dap-Hol-NH2(SEQ ID NO:21);
Cholesteryl-O—CO-Arg-Pro-Arg-Nva-Tyr-Dap-Hol-NH₂(SEQ ID NO:22);
Cholesteryl-O—CO-Arg-Pro-Arg-Nva-Tyr-Ala-Hol-NH₂(SEQ ID NO:23);
Cholesteryl-O—CO-Gly-Arg-Pro-Arg-Nva-Tyr-Ala-Hol-NH₂(SEQ ID NO :24);
Vitamin E-Succinate-Arg-Pro-Arg-Nva-Tyr-Dap-Hol-NH₂(SEQ ID NO:25);
H—(DArg)₉-Gly-Arg-Pro-Arg-Nva-Tyr-Ala-Hol-NH₂(SEQ ID NO:26 ); and
Cholesteryl-O—CO-Arg-Pro-Arg-Orn-Glu(Aminoethylsulfonamidoisoquinoline)-Ser-Phe-NH₂(SEQ ID NO:27).

Another aspect of the present invention is directed to pharmaceutical compositions comprising as an active ingredient novel peptide conjugates that are inhibitors of protein kinase and to methods for the preparation and use of pharmaceutical compositions comprising these novel inhibitors of protein kinases.

Another aspect of the present invention is directed to the use of pharmaceutical compositions comprising these peptide conjugates for production of medicaments useful for the treatment of diseases and disorders. The present invention discloses methods of treatment of disorders involving protein kinase, including but not limited to cancers, proliferation diseases, diabetes, cardiovascular pathologies, hemorrhagic shock, obesity, inflammatory diseases, diseases of the central nervous system, and autoimmune diseases. According to a specific embodiment of the present invention, the pharmaceutical compositions according to the present invention are useful for treatment of Hormone-Refractory-Prostate-Cancer or other cancer types associated or correlated with PKB levels including but not limited to: prostate cancer; breast cancer; ovarian cancer; colon cancer; renal cancer, melanoma and skin cancer; lung cancer; and hepatocarcinoma.

According to yet another embodiment the pharmaceutical compositions according to the present invention are administered in combination with other chemotherapeutic substances. The chemotherapy drugs, which could be administered together with the protein kinase inhibitors according to the present invention, may comprise any such agent known in the art, including but not limited to: mitoxantrone, topoisomerase inhibitors, spindle poison vincas: vinblastine, vincristine, vinorelbine (taxol), paclitaxel, docetaxel; alkylating agents: mechlorethamine, chlorambucil, cyclophosphamide, melphalan, ifosfamide; methotrexate; 6-mercaptopurine; 5-fluorouracil, cytarabine, gemcitabin; podophyllotoxins: etoposide, irinotecan, topotecan, dacarbazin; antibiotics: doxorubicin (adriamycin), bleomycin, mitomycin; nitrosoureas: carmustine (BCNU), lomustine, epirubicin, idarubicin, daunorubicin; inorganic ions: cisplatin, carboplatin; interferon, asparaginase; hormones: tamoxifen, leuprolide, flutamide, and megestrol acetate.

The present invention further provides methods for modulating the activity of protein kinases in a subject, comprising administering a therapeutically effective amount of a peptide conjugate that is a protein kinase inhibitor.

Essentially all of the uses known or envisioned in the prior art for protein kinase inhibitors, can be accomplished with the molecules of the present invention.

By way of exemplification, the compounds disclosed in the present invention were selected for inhibition of Protein kinase B. Using the preparations and methods disclosed herein it is possible to obtain compounds that inhibit the activity of other types of protein kinases. These and other features of the present invention will be better understood in relation to the figures, detailed description, examples and claims that follow.

### BRIEF DESCRIPTION OF THE DRAWINGS

FIG. 1 is a graph that illustrates the viability of PC3 cells after treatment with PTRs 6164 and PTR 6244 as measured in growth inhibition assay.

FIG. 2 is a graph that illustrates the viability of LNCaP cells after treatment with PTRs 6180, 6198 and 6244, as measured in growth inhibition assay.

FIG. 3 is a graph that illustrates the viability of LNCAP cells after treatment with PTRs 6252, 6260 and 6244, as measured in growth inhibition assay.

FIG. 4 is a Western Blot analysis of AKT (S473) and GSK3 (S9/21) phosphorylation in LNCaP cells treated with PTR 6072, 6196 and 6164.

FIG. 5 is a graph that illustrates the biostability of PTR 6164 in plasma as measured in HPLC.

FIG. 6 is a graph that illustrates the apoptosis induced in Jurkat cells by PTR 6164 measured using Annexin-V staining and FACS analysis.

FIGS. 7A and 7B are graphs that illustrates cell growth inhibition induced by PKB inhibitors PTR 6164 (A) and 6260 (B) in cancer cells vs. normal blood cells.

FIG. 8 is a graph that illustrates the efficacy of in vivo study in mice bearing PC3 tumor xenograft using i.t. administration of PTR 6164.

FIG. 9 is a graph that illustrates the effect of systemic (i.p.) administration of PTR 6164 on growth of prostate cancer xenografts in mice.

FIG. 10 is a graph that illustrates the effect of treatment with PTR 6164 on apoptosis and mitosis in PC3 tumors growing in nude mice and measured in stained tumor sections.

FIGS. 11A and 11B are graphs that illustrates the in vitro selectivity of PTR 6320 in comparison to PTR 6164 in inhibition of protein kinase A vs. protein kinase B activity.

FIG. 12 is a graph that illustrates the cell death induced by PTR 6320 in prostate cancer cell lines vs. normal cells.

FIG. 13 is a Western blot analysis of AKT and FKHR phosphorylation in LNCaP cells treated with PTR 6164, 6320 and 6344.

FIGS. 14A and 14B are Western blot analyses of AKT and FKHR phosphorylation in 786-O (A) and MDA468 (B) cells treated with PTR 6164 and 6320.

FIG. 15 is a graph that illustrates the induction of apoptosis in prostate cancer cells by caspase activity, induced in prostate cancer cell line (LNCaP) by PTR 6164 and PTR 6320.

FIGS. 16A to 16E are graphs that illustrate the cell death of prostate cancer cell lines following treatment with combination treatment of protein kinase inhibitors and known chemotherapy agents.

### DETAILED DESCRIPTION OF THE PREFERRED EMBODIMENTS

In the production and screening of potential PKB inhibitors of various types, it was surprisingly found that peptide and peptidomimetic conjugates according to the present invention possess improved pharmacological properties over previously known PKB inhibitors. Although previously described chimeric molecules comprising an ATP mimetic and a substrate mimetic, are more potent in PKB enzyme inhibition in cell-free assays, the novel peptide conjugates were found to be more selective toward PKB in comparison to PKA, and due to their cell permeability those components are capable of penetrating into cells and inhibiting intracellular events such as apoptosis and phosphorylation while the chimeric molecules do not inhibit PKB in cells. Therefore, the peptide conjugates of the present invention are more suitable for use as therapeutic agents.

In the present invention, independent substrate mimetic peptide inhibitors, which are selective inhibitors of PKB but have low activity in cells, were modified, in order to improve their pharmacological properties, and obtain cell permeable, serum stable inhibitors that retain selectivity to PKB over PKA.

When screening peptide conjugates according to the present invention it was surprisingly found that certain lipophilic moieties are preferred as cell-permeability enhancers than other. For example, peptide conjugates comprising cholesterol were significantly more active than similar compounds comprising myristoyl or lauryl.

In the present invention, core peptidomimetic compounds having in vitro and in vivo PKB inhibitory effect were identified. The most potent and selective one was optimized to achieve new compounds that are about five times more potent in all cellular assays, and are remarkably more selective. The new compounds induce cell death and apoptosis at 1-2 μM in prostate cancer cell lines, but are safe to normal cells at 40 μM. They induce apoptosis in cancer cells at 3 μM and show inhibition of downstream substrates of PKB by western blot at 3-5 μM. The utility of the compositions according to the invention can be established by means of various assays as are well known in the art. The preferred compounds of the present invention were found to be active in a panel of in-vitro assays, in inhibiting the activity of protein kinases and in induction of cell death and apoptosis in cancer cells but not in normal cells. In addition, selected compounds, which exhibit high activity in vitro are tested in vivo for evaluation of their effect on tumor growth, tumor regression, and potential synergistic effects with chemotherapy agents.

Selected compounds according to the present invention are peptide-based, substrate mimetic inhibitors of PKB that are stable in plasma for 6-24 hours, slowly metabolized by hepatic cells and are membrane permeable. These compounds are 10-200 times selective for PKB over related kinases, and were characterized in tissue culture as potent, selective inhibitors of this protein kinase. The inhibitors induce cell death specifically in prostate, breast and renal cancer cells, in which PKB is highly activated, but not in normal cells in which there is very little or no PKB activation. The inhibitors induce apoptosis in prostate cancer cells in the same concentrations that cell death is induced, while no cytotoxic death is observed at these concentrations by cell cycle analysis. Furthermore, the inhibitors decrease the phosphorylation of PKB's downstream substrates in prostate cancer cells.

Pharmaceutical compositions according to the present invention comprising pharmacologically active protein kinase inhibitors and a pharmaceutically acceptable excipient, carrier or diluent represent another embodiment of the invention, as do the methods for the treatment of a mammal in need thereof with a pharmaceutical composition comprising an effective amount of a protein kinase inhibitor according to the invention. Methods of treatment using the compositions of the invention are useful for therapy of cancers, proliferation diseases, diabetes, cardiovascular pathologies, hemorrhagic shock, obesity, inflammatory diseases, diseases of the central nervous system, and autoimmune diseases using such compositions.

The pharmaceutical compositions according to the present invention may be most preferably used for prevention and treatment of malignancies selected from the group of Hormone-Refractory-Prostate Cancer; Prostate cancer (Zin et al, Clin. Cancer Res.2001 7,2475-9); Breast Cancer (Perez-Tenorio and Stal, Brit. J. Cancer 2002 86, 540-45, Salh et al, Int. J. Cancer 2002 98,148-54); Ovarian cancer (Liu et al, Cancer Res. 1998 15, 2973-7); Colon cancer (Semba at al, Clin. Cancer. Res. 2002 8,1957-63); Melanoma and skin cancer (Walderman, Wecker and Diechmann, Melanoma Res. 2002 12, 45-50); Lung cancer(Zin et al, Clin. Cancer Res.2001 7,2475-9); and hepatocarcinoma (Fang et al, Eur. J. Biochem. 2001 268, 4513-9).

Additional specific types of cancers that can be treated using this invention include acute myelogenous leukemia, bladder, breast, cervical, cholangiocarcinoma, chronic myelogenous leukemia, colorectal, gastric sarcoma, glioma, leukemia, lung, lymphoma, melanoma, multiple myeloma, osteosarcoma, ovarian, pancreatic, prostate, stomach, or tumors at localized sites including inoperable tumors or in tumors where localized treatment of tumors would be beneficial, and solid tumors.

In addition, indications that may be treated using the pharmaceutical compositions of the present invention include any condition involving undesirable or uncontrolled cell proliferation, providing that protein kinases, and especially PKB, levels, are elevated, associated or correlated with the indication. Such indications include restenosis, benign tumors, a various types of cancers such as primary tumors and tumor metastasis, abnormal stimulation of endothelial cells (atherosclerosis), insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

Specific types of restenotic lesions that can be treated using the present invention include coronary, carotid, and cerebral lesions. Specific types of benign tumors that can be treated using the present invention include hemangiomas, acoustic neuromas, neurofibroma, trachomas and pyogenic granulomas.

Treatment of cell proliferation due to insults to body tissue during surgery may be possible for a variety of surgical procedures, including joint surgery, bowel surgery, and cheloid scarring. Diseases that produce fibrotic tissue include emphysema. Repetitive motion disorders that may be treated using the present invention include carpal tunnel syndrome. An example of cell proliferative disorders that may be treated using the invention is a bone tumor.

Abnormal angiogenesis that may be may be treated using this invention include those abnormal angiogenesis accompanying rheumatoid arthritis, psoriasis, diabetic retinopathy, and other ocular angiogenic diseases such as retinopathy of prematurity (retrolental fibroplastic), macular degeneration, corneal graft rejection, neuroscular glaucoma and Oster Webber syndrome.

The proliferative responses associated with organ transplantation that may be treated using this invention include those proliferative responses contributing to potential organ rejections or associated complications. Specifically, these proliferative responses may occur during transplantation of the heart, lung, liver, kidney, and other body organs or organ systems.

The pharmaceutical compositions according to the present invention advantageously comprise at least one protein kinase inhibitor. These pharmaceutical compositions may be administered by any suitable route of administration, including topically or systemically. Preferred modes of administration include but are not limited to parenteral routes such as intravenous and intramuscular injections, as well as via nasal or oral ingestion. As it is known to those skilled in the art the pharmaceutical compositions may be administered alone own or in conjunction with additional treatments for the conditions to be treated.

It was now found that simultaneous or sequential administration of both the anti PKB compounds according to the present invention and known chemotherapy drugs results in an increase in antitumour activity such that the anticancer effect of the combination is much larger than for either agent alone, and greatly exceeds the sum of effects of the individual agents. Therefore, the present application provides protein kinase inhibitors which may be used in combination therapy with any other agent known to be used for treatment of malignancies or other proliferative responses.

Terminology and Definitions

In the specification and in the claims the term “protein kinase” refers to a member of an enzyme superfamily which functions to phosphorylate one or more protein as described above.

As used herein and in the claims, the term “inhibitor” is interchangeably used to denote “antagonist” these terms define compositions which have the capability of decreasing certain enzyme activity or competing with the activity or function of a substrate of the enzyme.

As used herein and in the claims the term “chimeric compound” or “chimeric molecule” denotes an ATP mimic moiety conjugated to a PKB substrate mimetic part.

As used herein “peptide” indicates a sequence of amino acids linked by peptide bonds. The peptide analogs of this invention comprise a sequence of 4 to 25 amino acid residues, preferably 5 to 20 residues, more preferably 6 to 15 amino acids, each residue being characterized by having an amino and a carboxy terminus.

The term “peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “peptidomimetic” may be computer assisted.

The term “spacer” denotes a chemical moiety whose purpose is to link, covalently, a cell-permeability moiety and a peptide or peptidomimetic. The spacer may be used to allow distance between the cell-permeability moiety and the peptide, or the spacer is a chemical bond of any type. Linker denotes a direct chemical bond or a spacer.

The term “peptide analog” indicates molecule which has the amino acid sequence according to the invention except for one or more amino acid changes or one or more modification/replacement of an amide bond.

The term “core” in the context of the present invention refers to the peptidic segment or moiety of the protein kinase inhibitor which comprises peptide or peptidomimetic and is optionally attached to a cell-permeability enhancer.

“Permeability” refers to the ability of an agent or substance to penetrate, pervade, or diffuse through a barrier, membrane, or a skin layer. A “cell permeability” or a “cell-penetration” moiety refers to any molecule known in the art which is able to facilitate or enhance penetration of molecules through membranes. Non-limitative examples include: hydrophobic moieties such as lipids, fatty acids, steroids and bulky aromatic or aliphatic compounds; moieties which may have cell-membrane receptors or carriers, such as steroids, vitamins and sugars, natural and non-natural amino acids and transporter peptides. Examples for lipidic moieties which may be used according to the present invention: Lipofectamine, Transfectace, Transfectam, Cytofectin, DMRIE, DLRIE, GAP-DLRIE, DOTAP, DOPE, DMEAP, DODMP, DOPC, DDAB, DOSPA, EDLPC, EDMPC, DPH, TMADPH, CTAB, lysyl-PE, DC-Cho, -alanyl cholesterol; DCGS, DPPES, DCPE, DMAP, DMPE, DOGS, DOHME, DPEPC, Pluronic, Tween, BRIJ, plasmalogen, phosphatidylethanolamine, phosphatidylcholine, glycerol-3 -ethylphosphatidylcholine, dimethyl ammonium propane, trimethyl ammonium propane, diethylammonium propane, triethylammonium propane, dimethyldioctadecylammonium bromide, a sphingolipid, sphingomyelin, a lysolipid, a glycolipid, a sulfatide, a glycosphingolipid, cholesterol, cholesterol ester, cholesterol salt, oil, N-succinyldioleoylphosphatidylethanolamine, 1,2-dioleoyl-sn-glycerol, 1,3 -dipalmitoyl-2-succinylglycerol, 1,2-dipalmitoyl-sn-3 -succinylglycerol, 1 -hexadecyl-2-palmitoylglycerophosphatidylethanolamine, palmitoylhomocysteine, N,N′-Bis (dodecyaminocarbonylmethylene)-N,N′-bis((-N,N,N-trimethylammoniumethyl-ami nocarbonylmethylene)ethylenediamine tetraiodide; N,N″-Bis(hexadecylaminocarbonylmethylene)-N,N′, N″-tris((-N,N,N-trimethylammonium -ethylaminocarbonylmethylenediethylenetri amine hexaiodide; NN′-Bis(dodecylaminocarbonylmethylene)-N,N″-bis((-N,N,N-trimethylammonium ethylaminocarbonylmethylene)cyclohexylene-1,4-diamine tetraiodide; 1,7,7-tetra-((-N,N,N,N -tetramethylammoniumethylamino-carbonylmethylene)-3-hexadecylaminocarbonyl-methylene -1,3,7-triaazaheptane heptaiodide; N,N,N′,N′-tetra((-N,N,N-trimethylammonium -ethylaminocarbonylmethylene)-N′-(1,2-dioleoylglycero-3-phosphoethanolamino carbonylmethylene)diethylenetriamine tetraiodide; dioleoylphosphatidylethanolamine, a fatty acid, a lysolipid, phosphatidylcholine, phosphatidylethanolamine, phosphatidylserine, phosphatidylglycerol, phosphatidylinositol, a sphingolipid, a glycolipid, a glucolipid, a sulfatide, a glycosphingolipid, phosphatidic acid, palmitic acid, stearic acid, arachidonic acid, oleic acid, a lipid bearing a polymer, a lipid bearing a sulfonated saccharide, cholesterol, tocopherol hemisuccinate, a lipid with an ether-linked fatty acid, a lipid with an ester-linked fatty acid, a polymerized lipid, diacetyl phosphate, stearylamine, cardiolipin, a phospholipid with a fatty acid of 6-8 carbons in length, a phospholipid with asymmetric acyl chains, 6-(5-cholesten-3b-yloxy) -1-thio-b-D-galactopyranoside, digalactosyldiglyceride, 6-(5-cholesten-3b-yloxy)hexyl-6-amino -6-deoxy-1 -thio-b-D-galactopyranoside, 6-(5 -cholesten-3b-yloxy)hexyl-6-amino-6-deoxyl-1 -thio-a-D-mannopyranoside, 12-(((7′-diethylamino-coumarin-3 -yl)carbonyl)methylamino)-octadecanoic acid; N-[12-(((7′-diethylaminocoumarin-3-yl)carbonyl)methyl-amino) octadecanoyl]-2-aminopalmitic acid; cholesteryl)4′-trimethyl-ammonio)butanoate; N -succinyldioleoyl-phosphatidylethanolamine; 1,2-dioleoyl-sn-glycerol; 1,2-dipalmitoyl-sn-3 -succinyl-glycerol; 1,3-dipalmitoyl-2-succinylglycerol, 1 -hexadecyl-2-palmitoylglycero -phosphoethanolamine, and palmitoylhomocysteine.

In the specification and in the claims the term “therapeutically effective amount” refers to the amount of protein kinase inhibitor or composition comprising same to administer to a host to achieve the desired results for the indications described herein, such as but not limited of cancers, diabetes, cardiovascular pathologies, hemorrhagic shock, obesity, inflammatory diseases, diseases of the central nervous system, and auto immune diseases.

As used herein, “cancer” and “cancerous” refer to any malignant proliferation of cells in a mammal.

When two compounds are administered in combination or used in combination therapy according to the invention the term “combination” in this context means that the drugs are given contemporaneously, either simultaneously or sequentially. This term is exchangeable with the term “coadministration which in the context of this invention is defined to mean the administration of more than one therapeutic in the course of a coordinated treatment to achieve an improved clinical outcome. Such coadministration may also be coextensive, that is, occurring during overlapping periods of time.

The co-administration of a tyrosine kinase inhibitor and another agent can be by concurrent administration of separate formulations, i.e., a tyrosine kinase formulation and another agent formulation. Administration of separate formulations is “concurrent” if the timing of their administration is such that the pharmacological activities of the tyrosine kinase inhibitor and the other agent occur simultaneously in the mammal undergoing treatment.

In some embodiments of the invention, co-administration of a tyrosine kinase inhibitor and another agent is accomplished by formulating them into a single composition.

Certain abbreviations are used herein to describe this invention and the manner of making and using it. For instance, ATP refers to adenosine three phosphate, BSA refers to bovine serum albumin, BTC refers to bis-(trichloromethyl)carbonate or triphosgene, DIEA refers to diisopropyl-ethyl amine, DMF refers to dimethyl formamide, EDT refers to ethanedithiol, EDTA refers to ethylene diamine tetra acetate, ELISA refers to enzyme linked immuno sorbent assay, EGF refers to epithelial growth factor, FACS refers to fluorescence assisted cell sorter, FKHR refers to forkhead, GSK3 refers to glycogen synthase kinase 3, HA refers to hemagglutinin, HBTU refers to 1-hydroxybenztriazolyl tetramethyl-uronium, HEPES refers to 4-(2-hydroxyethyl)-1-piperazineethanesulfonic acid, HOBT refers to 1-hydroxybenzotriazole, HRPC refers to Hormone-Refractory-Prostate Cancer, IGF refers to insulin growth factor, MOPS refers to 4-morpholinepropanesulfonic acid, MPS refers to multiple parallel synthesis, NMP refers to N-methyl formamide, MTD refers to maximal tolerated dose, PBS refers to phosphate buffer saline, PKA refers to protein kinase A, PKB refers to protein kinase B, PKC refers to protein kinase C, rpm refers to rounds per minute, SAR refers to structure-activity relationship, THF refers to tetrahydrofuran, TIS refers to tri-isopropyl-silane, TFA refers to trifluoric acetic acid.

Chemistry:

Preferred peptides according to the present invention may be synthesized using any method known in the art, including peptidomimetic methodologies. These methods include solid phase as well as solution phase synthesis methods. The conjugation of the peptidic and permeability moieties may be performed using any methods known in the art, either by solid phase or solution phase chemistry. Non-limiting examples for these methods are described hereby. Some of the preferred compounds of the present invention may conveniently be prepared using solution phase synthesis methods. Other methods known in the art to prepare compounds like those of the present invention, can be used and are comprised in the scope of the present invention.

The amino acids used in this invention are those which are available commercially or are available by routine synthetic methods. Certain residues may require special methods for incorporation into the peptide, and either sequential, divergent and convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

List of non limiting examples of non-coded amino acids: Abu refers to 2-aminobutyric acid, Ahx6 refers to aminohexanoic acid, Ape5 refers to aminopentanoic acid, ArgOl refers to argininol, bAla refers to β-Alanine, Bpa refers to 4-Benzoylphenylalanine, Bip refers to Beta-(4-biphenyl)-alanine, Dab refers to diaminobutyric acid, Dap refers to Diaminopropionic acid, Dim refers to Dimethoxyphenylalanine, Dpr refers to Diaminopropionic acid, Hol refers to homoleucine, HPhe refers to Homophenylalanine, GABA refers to gamma aminobutyric acid, GlyNH₂ refers to Aminoglycine, Nle refers to Norleucine, Nva refers to Norvaline, Orn refers to Ornithine, PheCarboxy refers to para carboxy Phenylalanine, PheCl refers to para chloro Phenylalanine, PheF refers to para fluoro Phenylalanine, PheMe refers to para methyl Phenylalanine, PheNH₂ refers to para amino Phenylalanine, PheNO₂ refers to para nitro Phenylalanine, Phg refers to Phenylglycine, Thi refers to Thienylalanine.

Pharmacology

Apart from other considerations, the fact that the novel active ingredients of the invention are peptides, peptide analogs or peptidomimetics, dictates that the formulation be suitable for delivery of these type of compounds. Clearly, peptides are less suitable for oral administration due to susceptibility to digestion by gastric acids or intestinal enzymes. The preferred routes of administration of peptides are intra-articular, intravenous, intramuscular, subcutaneous, intradermal, or intrathecal. A more preferred route is by direct injection at or near the site of disorder or disease. However, some of the compounds of the present invention were proved to be highly resistance to metabolic degradation in addition to their ability to cross cell membrane. These properties make them potentially suitable for oral administration

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for the chosen route of administration. For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

In one embodiment, the invention provides a method of killing a cell (e.g., a targeted cell) by co-administering protein kinase inhibitor and a cytotoxic agent to the cell. In addition, any period of pretreatment can be employed in the inventive method; the exact period of pretreatment will vary depending upon the application for the inventive method. For example, in therapeutic applications, such pretreatment can be for as little as about a day to as long as about 5 days or more; more preferably, the pretreatment period is between about 2 and about 4 days (e.g., about 3 days). Following pretreatment, the inventive method involves administering a cytotoxic agent. However, in other embodiments, a glucocorticoid (e.g., cortisol, dexamethasone, hydrocortisone, methylprednisolone, prednisolone, prednisone, etc.), diphenhydramine, rantidine, antiemetic-ondasteron, or ganistron can be adjunctively administered, and such agents can be administered with the protein kinase inhibitor. The cytotoxic agent can be administered either alone or in combination with continued administration of the protein kinase inhibitor following pretreatment. While, according to certain embodiments, treatment ceases upon administration of the cytotoxic agent, it can be administered continuously for a period of time (e.g., periodically over several days) as desired.

The targeted cell can be solitary and isolated from other like cells (such as a single cell in culture or a metastatic or disseminated neoplastic cell in vivo), or the targeted cell can be a member of a collection of cells (e.g., within a tumor). Preferably, the cell is a neoplastic cell (e.g., a type of cell exhibiting uncontrolled proliferation, such as cancerous or transformed cells). Neoplastic cells can be isolated (e.g., a single cell in culture or a metastatic or disseminated neoplastic cell in vivo) or present in an agglomeration, either homogeneously or, in heterogeneous combination with other cell types (neoplastic or otherwise) in a tumor or other collection of cells. Where the cell is within a tumor, some embodiments of the present invention provides a method of retarding the growth of the tumor by administering protein kinase inhibitor to the tumor and subsequently administering a cytotoxic agent to the tumor. By virtue of the cytopathic effect on individual cells, the inventive method can reduce or substantially eliminate the number of cells added to the tumor mass over time. Preferably, the inventive method effects a reduction in the number of cells within a tumor, and, most preferably, the method leads to the partial or complete destruction of the tumor (e.g., via killing a portion or substantially all of the cells within the tumor).

Where the targeted cell is associated with a neoplastic disorder within a patient (e.g., a human), some embodiments of the invention provides a method of treating the patient by administering protein kinase inhibitor to the patient and subsequently administering a cytotoxic agent to the patient. This approach is effective in treating mammals bearing intact or disseminated cancer. For example, where the cells are disseminated cells (e.g., metastatic neoplasia), the cytopathic effects of the inventive method can reduce or substantially eliminate the potential for further spread of neoplastic cells throughout the patient, thereby also reducing. or minimizing the probability that such cells will proliferate to form novel tumors within the patient. Furthermore, by retarding the growth of tumors including neoplastic cells, the inventive method reduces the likelihood that cells from such tumors will eventually metastasize or disseminate. Of course, when the inventive method achieves actual reduction in tumor size (and especially elimination of the tumor), the method attenuates the pathogenic effects of such tumors within the patient. Another application is in high-dose chemotherapy requiring bone marrow transplant or reconstruction (e.g., to treat leukemic disorders) to reduce the likelihood that neoplastic cells will persist or successfully regrow.

In many instances, the pretreatment of cells or tumors with protein kinase inhibitor before treatment with the cytotoxic agent effects an additive and often synergistic degree of cell death. In this context, if the effect of two compounds administered together in vitro (at a given concentration) is greater than the sum of the effects of each compound administered individually (at the same concentration), then the two compounds are considered to act synergistically. Such synergy is often achieved with cytotoxic agents able to act against cells in the G₀-G₁ phase of the cell cycle.

Any cytotoxic agent can be employed in the context of the invention: as mentioned, many cytotoxic agents suitable for chemotherapy are known in the art. Such an agent can be, for example, any compound mediating cell death by any mechanism including, but not limited to, inhibition of metabolism or DNA synthesis, interference with cytoskeletal organization, destabilization or chemical modification of DNA, apoptosis, etc. For example, the cytotoxic agent can be an antimetabolite (e.g., 5-flourouricil (5-FU), methotrexate (MTX), fludarabine, etc.), an anti-microtubule agent (e.g., vincristine, vinblastine, taxanes (such as paclitaxel and docetaxel), etc.), an alkylating agent (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase-inhibitors (e.g., etoposide, camptothecins, etc.), or other cytotoxic agents (e.g., dexamethasone). The choice of cytotoxic agent depends upon the application of the inventive method. For research, any potential cytotoxic agent (even a novel cytotoxic agent) can be employed to study the effect of the toxin on cells or tumors pretreated with vitamin D (or a derivative). For therapeutic applications, the selection of a suitable cytotoxic agent will often depend upon parameters unique to a patient; however, selecting a regimen of cytotoxins for a given chemotherapeutic protocol is within the skill of the art.

For in vivo application, the appropriate dose of a given cytotoxic agent depends on the agent and its formulation, and it is well within the ordinary skill of the art to optimize dosage and formulation for a given patient. Thus, for example, such agents can be formulated for administration via oral, subcutaneous, parenteral, submucosal, intraveneous, or other suitable routes using standard methods of formulation. For example, carboplatin can be administered at daily dosages calculated to achieve an AUC (“area under the curve”) of from about 4 to about 15 (such as from about 5 to about 12), or even from about 6 to about 10. Typically, AUC is calculated using the Calvert formula, based on the glomerular filtration rate of creatinine (e.g., assessed by analyzing a plasma sample) (see, e.g., Martino et al., 1999, Anticancer Res., 19(6C), 5587-91). Paclitaxel can be employed at concentrations ranging from about 50 mg/m² to about 100 mg/m² (e.g., about 80 mg/m²). Where dexamethasone is employed, it can be used within patients at doses ranging between about 1 mg to about 10 mg (e.g., from about 2 mg to about 8 mg), and more particularly from about 4 mg to about 6 mg, particularly where the patient is human.

The dosage of the tyrosine kinase inhibitor according to the present invention is from 1 μg/kg to 1 g/kg of body weight per day. According to one embodiment, the dosage of the tyrosine kinase inhibitor is from 0.01 mg/kg to 100 mg/kg of body weight per day. The optimal dosage of the tyrosine kinase inhibitor will vary, depending on factors such as type and extent of progression of the prostate cancer, the overall health status of the patient, the potency of the tyrosine kinase inhibitor, and route of administration. Optimization of the tyrosine kinase dosage is within ordinary skill in the art.

Another embodiment of the invention provides a method of treating prostate cancer within a patient by adjunctively administrating protein kinase inhibitor and a glucocorticoid to the patient. Any protein kinase inhibitor and glucocorticoid can be employed in accordance with this aspect of the invention, many of which are discussed elsewhere herein and others are generally known in the art. Moreover, protein kinase inhibitor and the glucocorticoid are delivered to the patient by any appropriate method, some of which are set forth herein. Thus, they can be formulated into suitable preparations and delivered subcutaneously, intravenously, orally, etc., as appropriate. Also, for example, the glucocorticoid is administered to the patient concurrently, prior to, or following the administration of protein kinase inhibitor. One effective dosing schedule is to deliver between about 5 μg and about 25 μg/kg, protein kinase inhibitor daily on alternative days (e.g., between 2 and 4 days a week, such as Mon-Wed-Fri or Tues-Thus-Sat, etc.), and also between about 1 mg/kg and 20 mg/kg dexamethasone to a human patient also on alternative days. In such a regimen, the alternative days on which protein kinase inhibitor and on which the glucocorticoid are administered can be different, although preferably they are administered on the same days. Even more preferably, the glucocorticoid is administered once, by itself, prior to concurrent treatment. Of course, the treatment can continue for any desirable length of time, and it can be repeated, as appropriate to achieve the desired end results. Such results can include the attenuation of the progression of the prostate cancer, shrinkage of such tumors, or, desirably, remission of all symptoms. However, any degree of effect is considered a successful application of this method. A convenient method of assessing the efficacy of the method is to note the change in the concentration of prostate-specific antigen (PSA) within a patient. Typically, such a response is gauged by measuring the PSA levels over a period of time of about 6 weeks. Desirably, the method results in at least about a 50% decrease in PSA levels after 6 weeks of application, and more desirably at least about 80% reduction in PSA. Of course, the most desirable outcome is for the PSA levels to decrease to about normal levels.

General Synthetic Methods:

General Method for Synthesis of Chimeras in MPS Format

The following procedure describes the synthesis of peptides in 96 wells plate (MPS plate) at a scale of 6 μmol peptide per well, on Rink amide resin, using HBTU/HOBT for normal coupling.

One gram of rink amide 0.6 mmol/g was swelled overnight in NMP with gentle shaking. The resin was distributed into 96 wells plate (˜10 mg per well).

Fmoc deprotection performed by adding 500 μl of 25% piperidine solution in NMP to each well and mixing at 650 rpm for 15 min, the piperidine solution is removed by a pressure of nitrogen and another portion of piperidin solution is added and shacked for 15 min. Wash of resin after Fmoc deprotection and after couplings, performed by placing 600 μl NMP into each well, mixing for 2 min. and removing the NMP by nitrogen pressure. The washing procedure is repeated four times.

Regular coupling is performed by adding a solution of Fmoc protected amino acids (150 μl, 0.2 M) in HOBT/NMP to the resin, followed by addition of HBTU solution in DMF (150 μl, 0.2 M) and DIEA in NMP (150 μl, 0.4 M). The reaction vessel block is mixed at 650 rpm for 1 h and then removed by a pressure of nitrogen. This procedure is repeated once. The last amino acid used in the assembly is N-Boc protected. At the end of assembly allyl deprotection takes place (from Glu(OAllyl) or C-building unit) by placing 500 μl solution of Pd(PPhe3)4 (0.02M in chloroform containing 5% AcOH +2.5% NMM and mixing for 1 h. This procedure is repeated once. Wash of the resin after allyl deprotection performed by addition of 600 μl chloroform to each well and mixing for 5 min. The solvent is removed by nitrogen pressure. This wash is repeated for additional four times. The coupling of allyl protected linker to the peptide-resin is carried out by placing allyl protected linker (150μl, 0.2M in NMP) followed by addition of PyBoP (0.2M, in NMP) and DIEA (0.4 m, in NMP). The reaction vessel block is mixed for 1 h the solution is removed by a pressure of nitrogen. This procedure is repeated once. The resin after the coupling is washed by addition of 500 μl NMP to each well. Allyl removal from the linker is carried out followed the same procedure described above. After allyl deprotection, a solution of isoquinoline derivative (150 μl, 0.2 M in NMP) is added followed by addition of ByBoP (150 μl, 0.2M in NMP) and DIEA (150 μl, 0.4M in NMP). The reaction block is mixed for 2 h.

Wash of the resin after this coupling performed by addition of 600 μl NMP to each well and mixing for 2 min. The solvent is removed by nitrogen pressure. This wash is repeated for additional four times.

Cleavage and global deprotection are performed by transferring the resin from the reaction vessel block into a deep well microtiter plate (cleavage plate). To this plate 350 μl solution of 92.5% TFA, 2.5% H₂O, 2.5% TIS, 2.5% EDT is added. The plate is mixed at 1000 rpm for 1 h and then the TFA solution is evaporated to dryness.

Purification by Sep-Pak performed by dissolving the residue of the resin with the peptide in 900 μl solution A (0.1% TFA in water)+CH3CN 1:1 and applying on C-18 Sep-Pak column. This procedure is repeated once more. The plate is frozen in liquid nitrogen at least 15 min and the peptides are lyophilized.

Biological Screening Assays for Inhibition of Protein Kinase Activity:

A. Assays for Inhibition of Protein Kinases Activity in Cell Free System (in Vitro):

A1. PKA in Vitro Kinase Activity Assay

1.PKA enzyme was purchased from Promega. PKA activity is assayed on a 7-mer peptide, LRRASLG, known as kemptide. The assay is carried out in 96-well plates, in a final volume of 50 μl per well. The reaction mixture includes various concentrations of the inhibitor, 50 mM MOPS, 10 mM MgAc, 0.2 mg/ml BSA, 10 μM ATP, 20 μM Kemptide and 1 μCi γ³²P ATP. Reaction is started with addition of 15 μl of the catalytic subunit of PKA diluted in 0.1 mg/ml BSA, 0.4 U/well. Two blank wells without enzyme are included in every assay. The plates are agitated continuously at 30° C. for 10′. Reaction is stopped by addition of 12 μl 200 mM EDTA. 20 μl aliquots of the assay mixture are spotted onto 2 cm² phosphocellulose strips (e.g. Whatman P81) and immersed in 75 mM phosphoric acid (10 ml per sample). The phosphocellulose strips are washed 6 times. Washes are done in continuous swirling for 5 minutes. Last wash is in acetone. After air-drying the strips, radiation is measured by scintillation spectrometry.

2. Screening compounds for PKA inhibition was performed in 96-well plate using SPA beads, as described below for PKB with the following modifications; the enzyme substrate was 5 μM biotinylated-kemptide peptide (biotin-KLRRASLG). The kinase buffer was 50 mM MOPS pH 7, 0.2 mg/ml BSA, 10 mM Magnesium acetate. PKA (0.4 unit) diluted in 0.1 mg/ml BSA was added to each well.

A2. PKB in Vitro Kinase Activity Assay

1. PKB activity is assayed as described in Alessi et al. (FEBS Letters 399, 333, 1996) with the following modifications: instead of HA-PKB coupled to beads, soluble His-HA-PKB is used following precipitation on a Nickel column. The enzyme activity measurement is performed as described in the assay for PKA.

2. Screening compounds for PKB inhibition was performed in 96-well plate using method described previously (Kumar et al, BBA, 1526: 257-268, 2001) with modifications. Kinase reaction was carried out in final volume of 50 μl. Each well contained 2.5 μM of biotinylated-crosstide peptide (biotin-KGRPRTSSFA) in kinase buffer [50 mM Tris-HCl pH 7.5,10 mM MgCl₂ , 1 mM DTT and 0.1 mM sodium orthovanadate, 0.01% Triton X-100 and 2% dimethyl (Me₂SO)], His-PKB enzyme and the potential inhibitory compound. The reaction was started by adding 10 μl of 2 μM cold ATP and 0.25 μCi of [γ³³P]-ATP in kinase buffer. The plates were incubated at 27° C. for 1 hr. At the end of the incubation the reaction was stopped by 200 μl of PBS containing 0.1% Triton X-100, 5 mM EDTA, 1 mM ATP) and 0.3 mg/ml of SPA beads (Amersham Pharmacia Biotech). After 15 min incubation at room temperature, the reaction mixtures were filtered using Packard GF/B 96-well plates. The plates were washed twice with 2M NaCl and 1% orthophosphoric followed by ethanol wash and 1 hr air-dry. The radioactivity was counted using microplate Packard Top Count.

A3. PKC in Vitro Kinase Assay.

PKC was obtained from Promega and assayed according to the manufacturer's instructions using a kit from the same manufacturer, in the presence and absence of phospholipids. The activity of PKC was determined by subtracting the activity in the absence of phospholipids from that in the presence of phospholipids. The concentration of the ATP in the assay was 10 μM (Km for ATP=50 μM).

B. Assays for Inhibition of PKB Activity in Intact Cells:

Several cancer cell lines were used to determine the activity of PKB inhibitor compounds in intact cells. The human prostate carcinoma cell lines, PC-3 and LNCaP. The human acute T cells leukemia cell line, Jurkat. Human breast carcinoma cell lines: MCF-7 and MDA468 and renal adenocarcinoma cell 786-O. LNCaP,MDA468, 786-O and Jurkat cell lines express high basal level of activated PKB. PC-3 expresses moderate level of activated PKB. MCF-7 expresses low but inducible level of activated PKB. Control cells are PBLs, normal peripheral blood lymphocytes which obtained from normal donors (blood bank) and MCF10F which is a non tumorogenic breast cell line. The control cells are used to compare the effect of the protein kinase inhibitors on the tested cancer cells and the normal cells.

B1. Assays for Detection of Apoptosis:

Peptide conjugates, which are active in enzyme inhibition assays were tested in cells for induction of apoptosis of cancer cell lines. Apoptosis was assayed at least by two methods in each cell line. Cells were seeded at the appropriate plates for each method, treated with or without the inhibitory compounds for different time points and analyzed by one of the below methods.

a. Annexin-V Staining

This assay identifies the early event of phosphotidyl-serine presentation on cell membrane. Cells were assayed for apoptosis using the Annexin-V (Bender medsystems). Cells were seeded in 6-well plates (0.3×10⁶/well), and washed twice with PBS, 24 hrs after treatment with the inhibitory compounds, and resuspended in Annexin-V binding buffer (10 M Hepes/NaOH pH 7.4, 140 mM NaCl and 2.5 mM CaCl₂). Annexin-V was diluted 1:40 and added to each sample with 0.2 nM Propidium Iodide (PI). 0.5×10⁶ cells were taken per sample and analyzed by FACS.

b. Caspases Activity.

This assay indicates very early events of apoptosis. Caspases (1, 8, 9, 5, 7, 3, 6, 4, and 2) activity was assayed according to the manufacturer's instructions using the CaspaTag Caspase activity kit (Intergene), 24 hrs after treatment with the inhibitory compounds. Briefly, 10⁶ of suspended cells/ sample were labeled with 10 μl of 30× working dilution FAM-peptide-FMK -Fluorescein and incubated for 1 hr at 37° C. under 5% CO₂. Samples were washed 3 times with 1× working dilution wash buffer and the cell pellets were resuspended with 700 μl of the same buffer. 2 μl of 0.2 nM propidium iodide solution was added and caspases activity was determined by FACS analysis.

c. DNA Fragmentation Measurement.

DNA fragmentation is a late event in the apoptosis cascade. DNA fragmentation was measured according to the manufacturer's instructions using the In situ cell death detection kit (Roche), 72 hrs after treatment with the inhibitory compounds. Briefly, 2×10⁶ of adherent cells/ sample were trypsinized, washed twice with PBS, and replaced in 96 well plates. Then, the samples were fixed with 2% Paraformaldehyde in PBS at room temperature for 1 hr, washed with PBS and resuspended with permeabilization solution for 2 min on ice. Cells were washed twice with PBS, and labeled with TUNEL reaction mixture containing labeling solution and TdT enzyme solution for lhr at 37° C. Samples were washed again with PBS and analyzed by FACS.

B2. Growth Inhibition Assays:

Selected peptide conjugates, which were found active in the enzyme-inhibition assays were screened for their ability to inhibit growth of tumor cell lines. Screening for inhibitory compounds was done, initially, at concentration of 50 μM. Active compounds from the first screening were further tested at different concentrations (50, 25, 12.5, 6.25, 3.125 and 1.56 μM) in order to determine their IC₅₀. Growth inhibition was tested using two methods: A. staining of viable cells with methylene blue, B. incorporation of ³H-thymidine. For both methods cells were grown in 96-well plates: LNCaP, 5000 cell per well for 72 hours, PC3, 5000 cells per well for 48 hours Jurkat, MDA468, and 786-O, 2500, 5000 and 1000 cells per well respectively for 24 hours, before tested compounds were added. The assays were done in triplicates for one to six days.

a. Staining Viable Cells with Methylene Blue:

Cells were fixed by 0.5% glutardialdehyde followed by staining with 1% methylene blue in borate buffer (Sigma) for one hour. Cells then washed few times with distilled water, air dried and the color was extracted by adding 0.1 M HCl for one hour at 37° C. Quantitation of color intensity was performed by measurement of the optical density at 600 nm by ELISA reader.

b. Incorporation of ³H-thymidine:

At the appropriate time in culture 1uci of 3H-thymidine (stock of 5 Ci/mmole, Amersham) was added to each well containing 100 μl of medium for 5 hours. At the end of the incubation the cells were washed few times with PBS using cell harvester (Packard, USA), air dried for few hours and 50 μl of scintillation liquid was added. The radioactivity was counted using microplate counter, Packard TopCount.

Fifty percent inhibitory concentration (IC₅₀) values were calculated using nonlinear regression in one site competition model with GraphPad Prism version 3.03 Windows (GraphPad Software, San Diego, USA)

B3. Inhibition of Phosphorylation of PKB and Downstream Substrates:

Peptide conjugates identified as inhibitors of PKB were further tested in cells for their ability to inhibit the phosphorylation of several PBK downstream substrates. The substrate GSK3 is associated with cell metabolism and cell cycle. Forkhead (FKHR) is directly associated with apoptosis. Inhibitory activity in these assays indicates also that the positive compounds penetrate into the cells.

Cells (2×10⁶) were seeded in 25 cm² flasks grown for 2 days at normal medium conditions. Inhibitory compounds were added for 24 hrs and analyzed for their effect on PKB phosphorylation at Ser473 residue and on the phosphorylation of specific substrates of PKB, GSK3 and FKHR. At the end of the treatment cells were lysed using lysis buffer (20 mM Tris-HCl pH 7.4, 150 mM NaCl, 0.5% Triton-x100, 25 mM NaF, 2 mM AEBSF, 1 mM sodium orthovanadate, 10 mM β-glycerophosphate, 1 μg/ml aprotonin and 5 μg/ml leupeptin). Equal amounts of cell protein were resolved by 10% SDS-PAGE and electroblotted to PVDF membranes. Western blot analysis was performed using antibodies against phospho-Akt1 (Ser473), phospho-GSK3α (Ser21) and phosphor-FKHR (Thr24), which were obtained from Cell Signaling Technology and against Akt1 (Upstate), GSK3α (Transduction Laboratories), and FKHR (Cell Signaling Technology).

When using the MCF-7 cell line, the cells were stimulated with 50 ng/ml IGF-1 (Sigma) for 10 min, after the addition of the inhibitory compounds, compare to cells which were only stimulated with IGF-1. The effect of the inhibitory compounds was analyzed as detailed above.

C. In Vivo Models for Evaluating Efficacy of PKB Inhibitors

The appropriate doses of compounds, which determined experimentally by acute and chronic toxicity studies, are injected to the tumor at various stages of its growth. Injection at early stages reflect the compound effect on tumor growth, injection into an established tumor determine its effect on regression. In addition, synergy studies, where the compounds are injected into the tumor along with a known chemotherapy agent, are performed to evaluate synergistic effects resulting from tumor increased sensitivity to chemotherapy due PKB inhibition leading to increased apoptosis.

The compounds are tested for their effect on tumor growth, in tumor xenografts derived from prostate cancer cell lines:

i. PC3 cells

ii. LNCAP cells

iii. MDA468

iiii. 786-O

The study is divided into two parts: determination of the maximal tolerated dose (MTD) and efficacy experiments.

C1. MTD Determination

Balb/C mice (male, 4-6 weeks old from Harlan Co. Israel) were used to determine the MTD.

Acute MTD determination: each compound was IV injected at several dosages and mice were observed for acute clinical signs for a period of 24 hours after injection in order to determined the acute MTD.

In addition, acute and chronic MTD were determined. As a first step, the acute MTD was determined after one IP injection of several dosages of 160, 80, 40, 20 and 10 mg/Kg body weight. For further testing the chronic MTD, mice were IP injected with 50%, 25% and 12.5% of the acute MTD daily for 3 consecutive weeks. Mice were daily monitored for general health status for the treatment period and 3 more additional weeks. At the end of the experiment full autopsy was preformed.

C2. Efficacy Study in Mice Bearing PC3 Tumor

a. PC3 cells (5×106) were injected subcutaneously in matrigel, into hip area of Nude male mice (Harlan Co., Israel). Tumor size was determined by caliper using the formula: Length ×(width²)×0.4. The tumors were allowed to grow to volume of about 50 mm 3 before the treatment was started. The appropriate doses of compounds were injected subcutaneously into the region surrounding the tumors (IT injection). The treatment was given 3 times a week; every other day for two weeks, and the tumor size was measured once every two days.

b. Human prostatic PC-3 cancer cells are grafted subcutaneously in 5 weeks old male nude Balb/c mice of approximately 20 g. Each mouse will receive 2.5×10⁶cells at Day 0. Intraperitoneal (i.p.) treatment is initiated when tumour volume reaches approximately 50 mm³. I.p. administration is highly acceptable in preliminary experiments, and exemplifies the stability in the blood and liver, and the distribution through the blood into the tumor. The control groups is treated with the vehicle 10% DMSO -90% 2-hydroxypropyl-beta-cyclodextrin at 1×10⁻²M in sterile saline. Tumour volume measurements with callipers (equation=length×width²×0.4) and body weights are recorded 3 times weekly on Mondays, Wednesdays and Fridays. Mice are monitored twice daily for general health status. Mice are sacrificed when the tumour mass in the respective vehicle treated groups reaches approximately 20% of the body weight or if this should not be obtained, 3 weeks after the end of the treatment. At the end of the experiment, the tumours of the 3 first sacrificed mice in each group are collected and embedded in paraffin blocks for further histological examination. The remaining tumours are collected and frozen in liquid nitrogen for western blot analysis.

### EXAMPLES

The following examples are intended to illustrate how to make and use the compounds and methods of this invention and are in no way to be construed as a limitation. Although the invention will now be described in conjunction with specific embodiments thereof, it is evident that many modifications and variations will be apparent to those skilled in the art. Accordingly, it is intended to embrace all such modifications and variations that fall within the spirit and broad scope of the appended claims.

### Example 1.

Design and Screening of Peptide and Peptidomimetic Conjugates

Three peptides and peptidomimetics were used as core molecules, and several types of modifications were applied, based on various conjugates. The core peptides are presented in the following table together with their protein kinase inhibition activity:

TABLE 1
Core peptide inhibitors

| PTR | | IC₅₀ μM | |
|---|---|---|---|
| No. | Sequence | PKB | PKA |
| 6154 | Arg-Pro-Arg-Nva-Tyr-Dap-Hol (SEQ ID NO: 7) | 0.9 | >50 |
| 6132 | Arg-Pro-Arg-Orn-Glu- | 0.02 | 0.012 |
| | (NH—(CH2)2—NH—SO₂—Isoquinoline)Ser- | | |
| | Phe (SEQ ID NO: 8) | | |
| 6184 | Arg-Pro-Arg-Nva-Tyr-Ala-Hol (SEQ ID NO: 9) | 0.3 | >50 |

The cell-permeable moieties used for conjugation to the core peptides in this example were:
1. Hydrophobic moieties such as fatty acids, steroids and bulky aromatic or aliphatic compounds.
2. Moieties which may have cell-membrane receptors or carriers, such as steroids, vitamins and sugars.
3. Known transporter peptides or amino acids.

The resultant peptide conjugates were tested for inhibition of protein kinase activity in various cell-free and cell-based assay, and the screening results indicated that some of the modifications induce stability and cell permeability, resulting in cellular activity of 8-20 μM in the relevant cell lines: Prostate cancer PC3 and LNCaP, T-cell leukemia Jurkat, and breast cancer MCF-7. In addition, some of the new conjugates were also 10-20 times selective for PKB over PKA in contrast to previously described chimeric compounds.
1. From the hydrophobic moiety conjugate family, cholesterol showed the best results, while other steroids, such as testosterone and lithocholic acid, did not show cellular activity at all. Also fatty acid conjugates showed very low cellular activity, 25-50 μM by PTR 6180 compared to 12 μM of the cholesterol PTR 6164. PTR 6164 may also be penetrating through a membrane receptor, or a combination of hydrophobicity considerations and membrane receptor/transporter. Other hydrophobic conjugates such as biotin and fluorescein (PTRs 6158 and 6182) showed low cell permeability.
2. From the family aimed to membrane receptors or transporters, the sugars showed very low cellular activity. Several vitamins that were tested were not cell active, except for the vitamin E conjugate which showed cellular activity of 15-20 μM.
3. From the peptide transporter conjugates, 96 compounds were tested all were very active in the in-vitro kinase assay and very selective 10-50 fold, however only few were active in cells. The selected 6 active conjugates show 8-20 μM activity in cells.

### Example 2.

Synthesis of PTRs 6154, 6184, 6180, 6244, and 6252.

One gram of Rink amide MBHA resin (0.64 mmol/g), were swelled in N-methylpyrrolidone (NMP) in a reaction vessel equipped with a sintered glass bottom and placed on a shaker. All the Fmoc protecting groups were removed by reaction with 20% piperidine in NMP (2 times 15 minutes, 10 ml each) followed by NMP wash (5 times two minutes, 15 ml each). Fmoc removal was monitored by ninhydrin test. The first amino acid was coupled to the resin by using 3 eq of the Fmoc protected amino acid+3 eq PyBroP+6 eq of DIEA in 7 ml NMP, reaction time 1.5 h. The couplings of the other Fmoc protected amino acids were carried out using 3 eq (1.92 mmol) of the Fmoc amino acid +PyBrop (3 equivalents, 1.92 mmol)+DIEA (6 equivalents, 3.84 mmol) in NMP (7 ml) for 1 hour at room temperature. Reaction completion was monitored by the qualitative ninhydrin test (Kaiser test). After each coupling, the peptide-resin was washed with NMP (5 times with 15 ml NMP, 2 minutes each).

In the end of the assembly, the peptide resin was washed with CH2C12 and dried under reduced pressure then cleaved from the resin by reaction with TFA 95%, water 2.5%, TIS (tri-isopropyl-silane) 2.5% , at 0° C. for 15 minutes and 1.5 hours at room temperature under argon. The mixture was filtered and the resin was washed with a small volume of TFA. The filtrate was placed in a rotary evaporator and all the volatile components were removed. An oily product was obtained. It was triturated with ether and the ether decanted, three times. A white powder was obtained. This crude product was dried under reduced pressure.
PTR 6154: Arg-Pro-Arg-Nva-Tyr-Dap-Hol-NH₂ (SEQ ID NO:7) 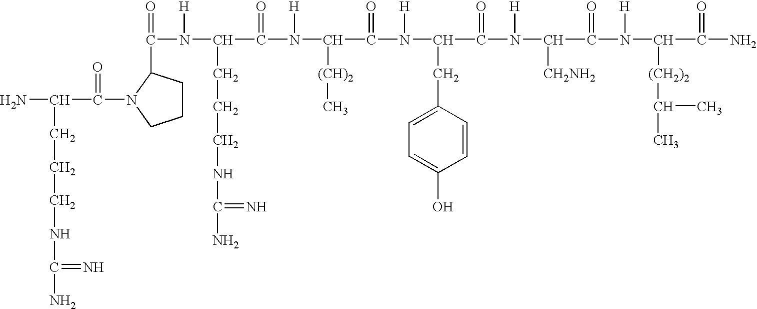
PTR 6184: Arg-Pro-Arg-Nva-Tyr-Ala-Hol-NH₂ (SEQ ID NO:9) 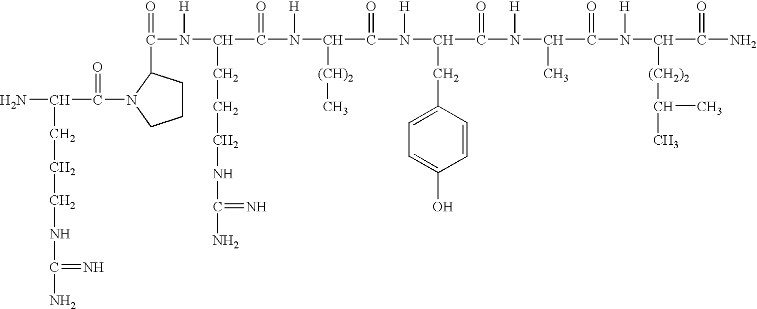
PTR 6180: Myristyl-Gly-Arg-Pro-Arg-Nva-Tyr-Dap-Hol-NH₂ (SEQ ID NO:28) 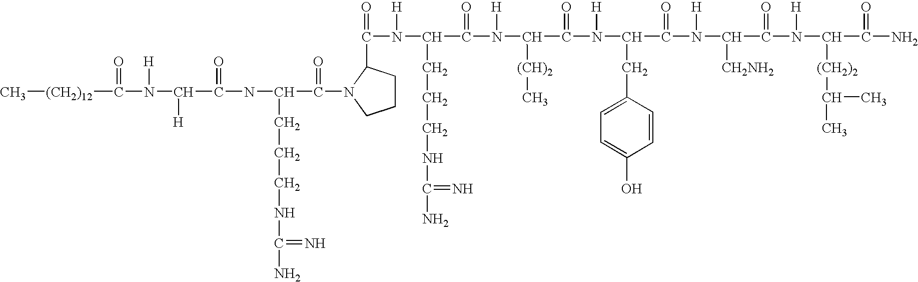
PTR 6244: Vitamin E succinate-Arg-Pro-Arg-Nva-Tyr-Dap-Hol-NH₂ (SEQ ID NO:25) 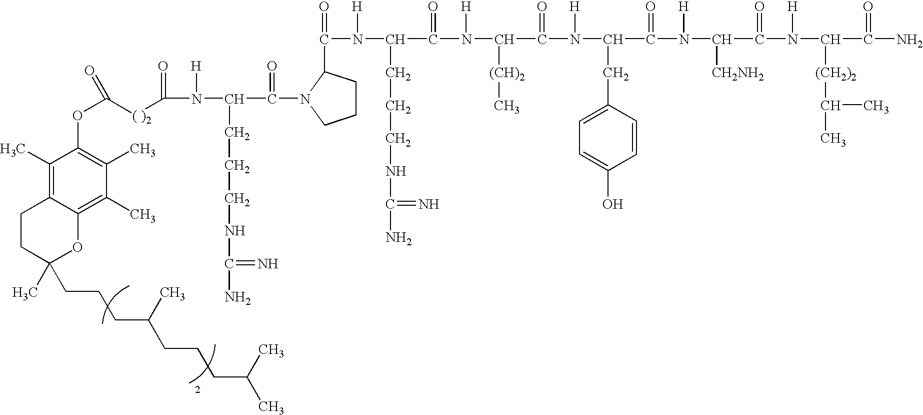
PTR 6252: (DArg)₉-Arg-Pro-Arg-Nva-Tyr-Ala-Hol-NH₂ (SEQ ID NO:26)

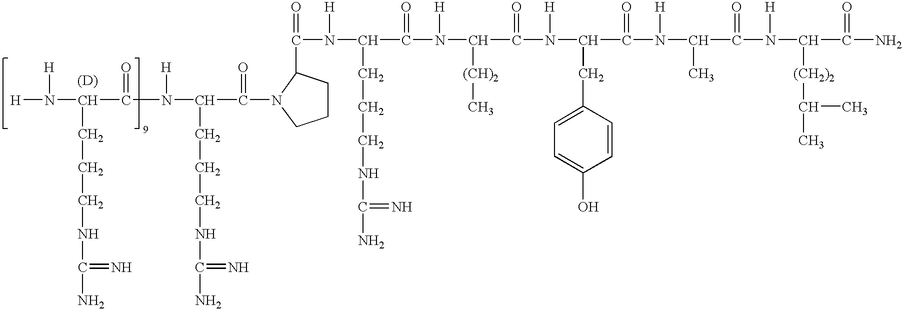

### Example 3.

Synthesis of PTR 6260

Five hundred mg of Rink amide MBHA resin (0.64 mmol/g) were swelled for 1.5 h in NMP in a reactor equipped with a sintered glass bottom, attached to a shaker. Fmoc was removed from the resin using 25% Piperidine in NMP (4 ml) twice for 15 min followed by careful wash, seven times with NMP (5 ml), for 2 min each. Assembly of Phe, Ser, Glu, Orn, Arg, Pro, Arg was accomplished by coupling cycles using Fmoc-Phe-OH, Fmoc-Ser(t-Bu)—OH, Fmoc-Glu(OAllyl)—OH, Fmoc-Orn(Boc)—OH, Fmoc-Arg(Pbf)—OH, and Fmoc-Pro—OH respectively. In each coupling cycle, the amino acid (3 equivalents) was dissolved in NMP and was activated with PyBroP (3 equivalents) and DIEA (6 equivalents). After coupling of Fmoc-Arg(Pbf)—OH at position 4, allyl deprotection took place using, Pd(PPh3)4 in solution of CH2C12 containing 5% AcOH and 2.5% NMM. The free acid was activated by 3 equivalents PyBoP, 6 equivalents DIEA in NMP and coupled with 3 eq of Ethylenediaminesulfonamido isoquinoline for 1 h. Following coupling, the peptide-resin was washed with NMP, then Fmoc was removed followed by coupling of Fmoc-Pro-OH and Fmoc-Arg(Pbf)—OH. The coupling of cholesterol to the N-terminal Arg was carried out by addition of 15 ml solution of dioxane: 1,3 -dichloropropane 1:2 containing cholesterol (5 eq)+BTC (1.66 eq)+collidine (15 eq), coupling time 1.5 h.

At the end of the synthesis the peptide was cleaved from the resin using 70% TFA, 7% TIS and 23% CH2C12 in a total volume of 10 ml cock-tail mixture for 15 min at 00C under Argon and then 1 h at room temperature. The solution was filtered through extract filter into polypropylene tube, the resin was washed with 5 ml solution of 70% TFA in CH2C12. The combined solution was evaporated to give oily residue, which on treatment with cold Et2O solidify. Centrifugation and decantation of the Et2O layer and treatment with additional portion of cold Et2O followed by centrifugation, decantation and drying the white solid under vacuum over night gave crude material denoted PTR 6260 having the following structure:
PTR 6260:
Cholesteryl-O—CO-Arg-Pro-Arg-Orn-Glu(Aminoethylsulfonamidoisoquinoline)-Ser-Phe-NH₂ (SEQ ID NO:27)

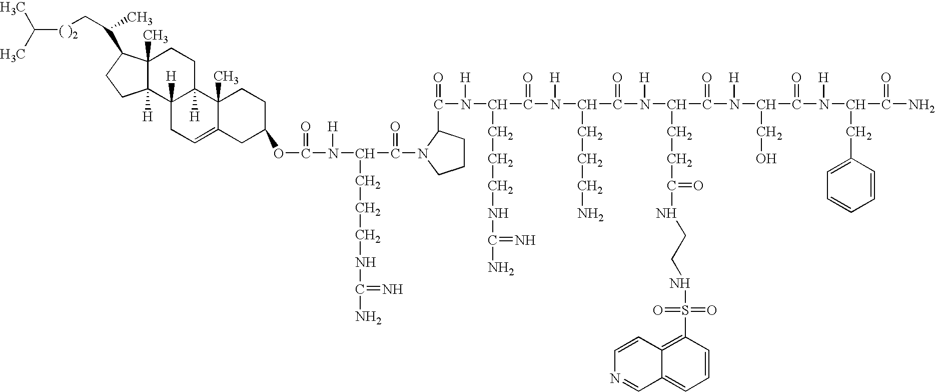

### Example 4

Synthesis of Peptides Conjugated to Cholesterol

One gram of Rink amide MBHA resin (0.64 mmol/g), was swelled in N-methylpyrrolidone (NMP) in a reaction vessel equipped with a sintered glass bottom and placed on a shaker. All the Fmoc protecting groups were removed by reaction with 20% piperidine in NMP (2 times 15 minutes, 10 ml each) followed by NMP wash (5 times two minutes, 15 ml each). Fmoc removal was monitored by ninhydrin test. The first amino acid was coupled to the resin by using 3 eq of the Fmoc protected amino acid+3 eq PyBroP+6 eq of DIEA in 7 ml NMP, reaction time 1.5 h. The couplings of the other Fmoc protected amino acids were carried out using 3 eq (1.92 mmol) of the Fmoc amino acid+PyBrop (3 equivalents, 1.92 mmol)+DIEA (6 equivalents, 3.84 mmol) in NMP (7 ml) for 1 hour at room temperature. Reaction completion was monitored by the qualitative ninhydrin test (Kaiser test). After each coupling, the peptide-resin was washed with NMP (5 times with 15 ml NMP, 2 minutes each). The coupling of cholesterol to the N-terminal Arg was carried out by addition of 15ml solution of dioxane: 1,3-dichloropropane 1:2 containing cholesterol (5 eq)+BTC (1.66 eq)+collidine (15 eq), coupling time 1.5 h.

At the end of the assembly, the peptide resin was washed with CH2C12 and dried under reduced pressure then cleaved from the resin by reaction with TFA 70%, water, TIS (tri-isopropyl-silane) 7%, CH2C12 23% , at 0° C. for 10 minutes and 50 min at room temperature under argon. The mixture was filtered and the resin was washed with a small volume of 70% TFA in CH₂Cl₂. The filtrate was placed in a rotary evaporator and all the volatile components were removed. An oily product was obtained. It was triturated with ether and the ether decanted, three times. A white powder was obtained. This crude product was dried under reduced pressure.
PTR 6164: Cholesteryl-O—CO-Arg-Pro-Arg-Nva-Tyr-Dap-Hol-NH₂ (SEQ ID NO:22) 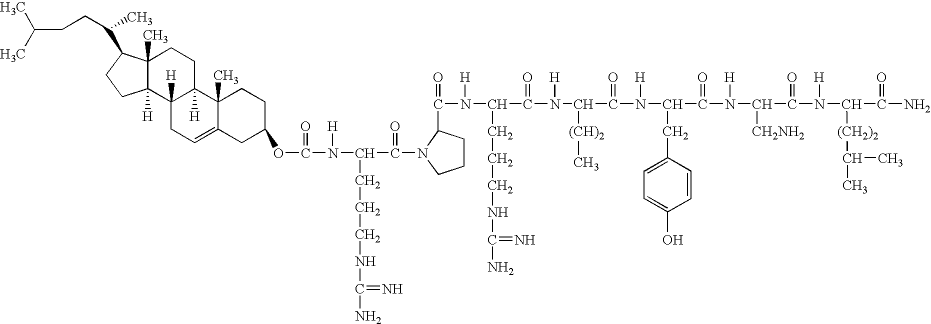
PTR 6196: Cholesteryl-O—CO-Arg-Pro-Arg-Nva-Tyr-Ala-Hol-NH₂ (SEQ ID NO:23) 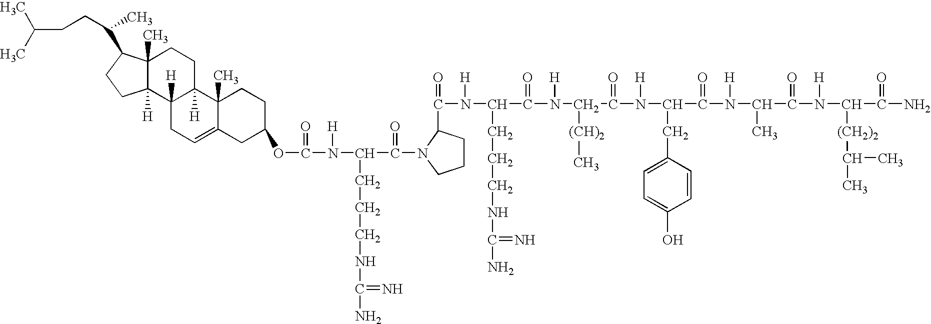
PTR 6198: Cholesteryl-O—CO-Gly-Arg-Pro-Arg-Nva-Tyr-Ala-Hol-NH₂ (SEQ ID NO:24)

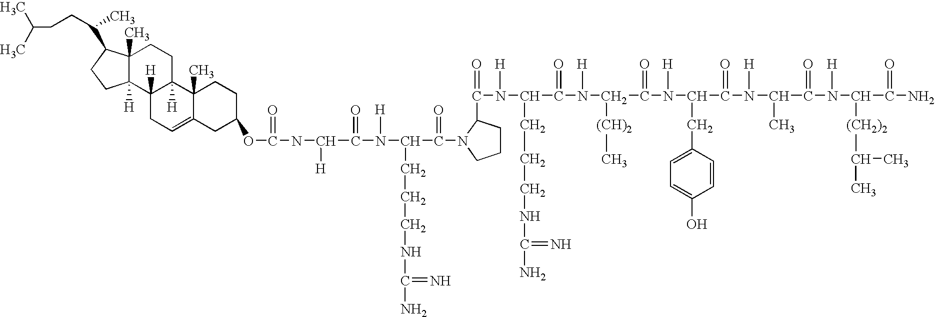

### Example 5

Peptides Conjugated to Transporter Peptides

2 describes the screening of peptide conjugates comprising transporter peptides. Peptides that were active also in cells, are shaded.

TABLE 2

| | % Inhibition | | |
|---|---|---|---|
| Peptide No. | | PKB activity | PC3 growth |

| MPS ab60025- | 10 μM | 1 μM | 50 μM |
|---|---|---|---|
| 2 | 92 | 60 | 60 |
| 4 | 96 | 80 | 52 |
| 17 | 99 | 98 | −3 |
| 20 | 95 | 88 | −3 |
| 22 | 93 | 78 | 3 |
| 23 | 98 | 99 | 0 |
| 24 | 96 | 85 | 11 |
| 25 | 72 | 4 | −7 |
| 27 | 41 | −9 | 3 |
| 29 | 99 | 99 | −2 |
| 30 | 93 | 85 | −8 |
| 33 | 99 | 100 | −5 |
| 35 | 98 | 98 | 6 |
| 36 | 99 | 98 | −1 |
| 38 | 98 | 86 | 0 |
| 41 | 99 | 97 | −5 |
| 42 | 97 | 70 | −5 |
| 43 | 99 | 93 | −5 |
| 44 | 98 | 92 | −11 |
| 45 | 99 | 99 | 10 |
| 46 | 97 | 90 | −3 |
| 49 | 99 | 97 | 37 |
| 50 | 98 | 93 | 30 |
| 54 | 97 | 95 | 39 |
| 58 | 90 | 82 | 21 |
| 60 | 77 | 23 | 7 |
| 66 | 96 | 93 | 39 |
| 68 | 88 | 61 | 26 |
| 69 | 99 | 99 | −5 |
| 70 | 85 | 40 | 15 |
| 72 | 77 | 21 | −1 |
| 78 | 96 | 82 | 4 |
| 79 | 99 | 98 | −14 |
| 80 | 91 | 66 | 9 |
| 81 | 98 | 92 | 19 |
| 82 | 92 | 78 | 50 |
| 83 | 98 | 93 | 12 |
| 84 | 97 | 81 | 11 |
| 86 | 90 | 71 | −7 |
| 88 | 95 | 83 | 1 |
| 89 | 99 | 98 | −20 |
| 90 | 82 | 43 | −16 |
| 93 | 99 | 96 | 28 |
| 94 | 92 | 76 | 20 |
| PTR 6252 | 93 | 65 | 65 |

Table 3 shows the IC₅₀ (μM) values of selected most active peptides from transporter 0025. Growth inhibition was measured according to assay B2 above. In vitro PKB inhibition was measured according to assay A2 above, PKB in vitro kinase activity assay in LNCap cells.

TABLE 3

| Peptide | | SEQ ID | Growth | in vitro PKB |
|---|---|---|---|---|
| AB60025- | Sequence | NO | inhibition | inhibition |
| 2 | Arg-Gln-Ile-Lys-Ile-Trp-Phe-Gln-Asn- | 29 | <12.5 | 1 |
| | Arg-Arg-Met-Lys-Trp-Lys-Lys-6154 | | | |
| 4 | Arg-Gln-Ile-Lys-Ile-Trp-Phe-Gln-Asn- | 30 | 10 | 0.2 |
| | Arg-Arg-Met-Lys-Trp-Lys-Lys-6184 | | | |
| 49 | 6154-(DLys)₁₀ | 31 | 10 | 0.04 |
| 82 | (DLys)₈-6154 | 32 | 10 | 0.4 |
| 50 | (DLys)₁₀-6154 | 33 | 13 | 0.08 |
| 93 | 6154-(DArg)₇ | 34 | 15 | 0.04 |
| 54 | Ahx6-DArg-DArg-DArg-DArg-DGln- | 35 | 20 | 0.04 |
| | DArg-DArg-DLys-DLys-DArg-6154 | | | |
| 94 | (DArg)₇-6154 | 36 | 25 | 0.4 |
| 6252 | (DArg)₉-6184 | 26 | 20 | 0.8 |

### Example 6

Activity of Peptide Conjugates

The activity and selectivity of selected most active peptide conjugates is presented in table 4.

TABLE 4

| | | IC₅₀ μM | | | | | |
|---|---|---|---|---|---|---|---|
| | SEQ | Kinase | | Growth | | | |
| | ID | assays | | inhibition in Cell lines | | | |
| PTR | NO: | PKB | PKA | PC3 | LNCAP | JURKAT | Structure |
| 6164 | 22 | 0.7 | 10 | 16 | 12 | 12 | Cholesteryl- |
| | | | | | | | O—CO-6154 |
| 6180 | 28 | 0.9 | 20 | 32 | 40 | 50 | Myristoyl- |
| | | | | | | | Gly-6154 |
| 6196 | 23 | 0.5 | 5 | 50 | 50 | 50 | Cholesteryl- |
| | | | | | | | O—CO-6184 |
| 6198 | 24 | 0.5 | 5 | 50 | 45 | 40 | Cholesteryl- |
| | | | | | | | O—CO- |
| | | | | | | | Gly-6184 |
| 6244 | 25 | 3 | 10 | 20 | 15 | 15 | Vitamin E- |
| | | | | | | | Succinate-6154 |
| 6252 | 26 | 0.8 | 10 | ND | 20 | ND | D(Arg)g-6184 |
| 6260 | 27 | 1 | >5 | ND | 8.5 | ND | Cholesteryl- |
| | | | | | | | O—CO-6132 |

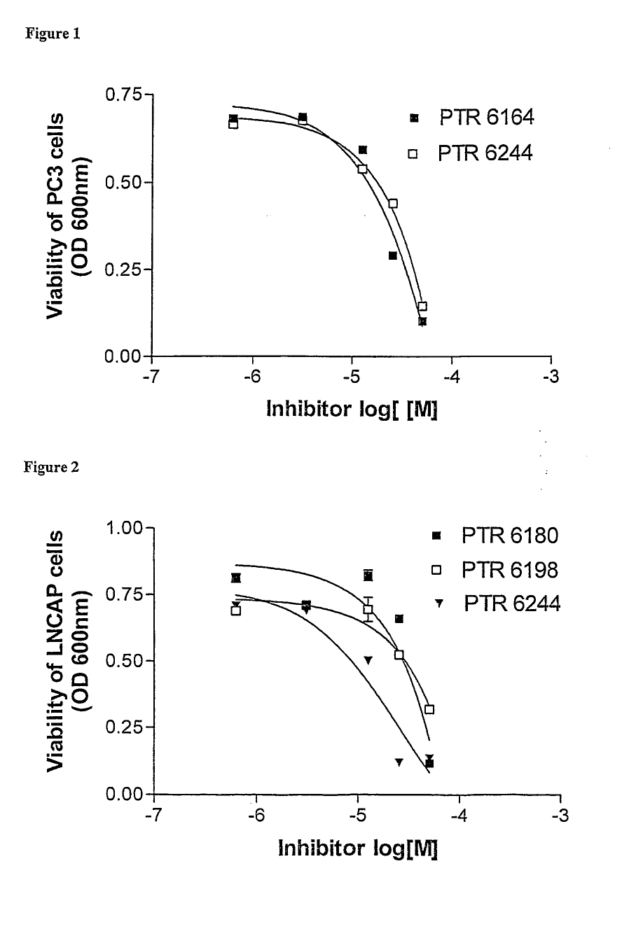

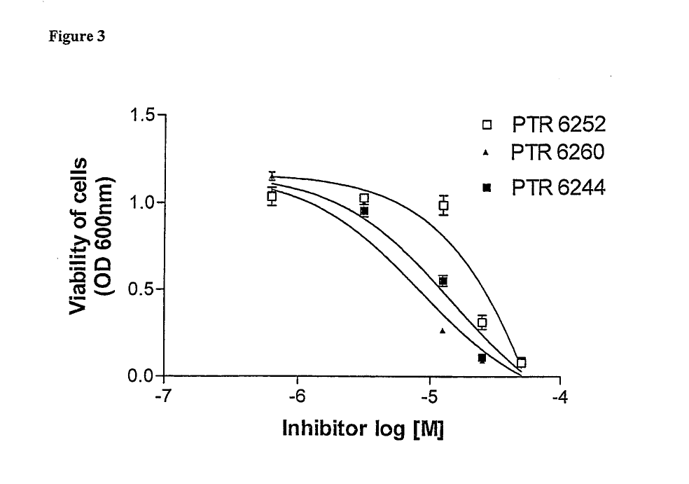

Growth inhibition curves for these compounds, determining the ability of the compounds to cause cell death in cancer cells, are presented in FIGS. 1-3:
FIG. 1 depicts viability of PC3 cells after treatment with PTRs 6164 and 6244;
FIG. 2 describes viability of LNCaP cells after treatment with PTRs 6180, 6198 and 6244;
FIG. 3 shows liability of LNCaP cells after treatment with PTRs 6252, 6260 and 6244.

High inhibitory activity as described indicates that the compounds are effective in inducing cell death or growth arrest in tumors, important in the evaluation of compounds as efficient anticancer drugs.

Table 5 represent selected peptide conjugates that were not active in cells.

TABLE 5

| | | | IC₅₀ μM | | | | |
|---|---|---|---|---|---|---|---|
| | | | Kinase | | Growth inhibition in Cell | | |
| | | | assays | | lines | | |
| PTR | SEQ ID NO: | Structure | PKB | PKA | PC3 | LNCAP | Jurkat |
| 6158 | 37 | Biotinyl-6154 | 1 | >100 | >50 | >50 | >50 |
| 6160 | 38 | Lauryl-6154 | 8 | 50 | >50 | >50 | >50 |
| 6182 | 39 | S-carbofluorescin- | 3 | >10 | >50 | >50 | >50 |
| | | 6154 | | | | | |
| 6192 | 40 | Lithocholyl-Gly-6184 | 2.5 | >10 | >50 | >50 | >50 |
| 6194 | 41 | Lithocholyl-6184 | 2 | >10 | >50 | >50 | >50 |
| 6200 | 42 | H-1-adamantyl-6184 | 0.3 | >10 | >50 | >50 | >50 |
| 6202 | 43 | H-1-adamantyl-CH2—CO- | 0.3 | >10 | >50 | >50 | >50 |
| | | 6184 | | | | | |
| 6212 | 44 | Testosterone-yl-O—CO- | 7 | | >50 | >50 | >50 |
| | | 6154 | | | | | |
| 6214 | 45 | Galactopyranosyl-O—CO- | 10 | | >50 | >50 | >50 |
| | | 6154 | | | | | |
| 6218 | 46 | Lithocholyl-Gly-6154 | 0.8 | | >50 | >50 | >50 |
| 6220 | 47 | Lithocholyl-6154 | 2 | | >50 | >50 | >50 |
| 6224 | 48 | Galactopyranosyl-O—CO- | 5 | 8 | >50 | >50 | >50 |
| | | 6184 | | | | | |
| 6226 | 49 | Testosterone-yl-O—CO- | 3 | | >50 | >50 | >50 |
| | | 6184 | | | | | |
| 6228 | 50 | VitB6-6184 | 2 | | >50 | >50 | >50 |
| 6232 | 51 | VitB6-6154 | 1 | | >50 | >50 | >50 |
| 6242 | 52 | VitE-Succinate-6184 | 1 | | >50 | 50 | 50 |

### Example 7

Further Evaluation of the Inhibition Activity of the Peptide Conjugates in Cells.

The downstream substrate phosphorylation assays are aimed to evaluate if the inhibitors are working in the PKB pathway. PKB phosphorylates several proteins involved in cell cycle, cell metabolism and apoptosis, and inhibition of the enzyme in the cell, results in decrease in the phosphorylation of its substrates. In these studies the phosphorylation of GSK3 (associated with cell metabolism) and FKHR (forkhead, associated directly with apoptosis), were examined. The results show a significant decrease in the phosphorylation of these substrate caused by exposure of the cancer cells to selected peptide conjugates according to the present invention.

PKB is a central player in apoptosis of cancer cells. It sends a “survival signal” that prevents cancer cells from performing programmed cell death. Its inhibition, therefore, result in cell death through apoptosis. The apoptosis assays are aimed to determine that the cancer cells exposed to our PKB inhibitors, are dying as a result of programmed cell death. This implies that the effect of the inhibitors is not simply cytotoxicity, but an apoptotic process induced by inhibition of PKB. Since apoptosis is a complex process, three assays that target different steps are used: 1. Caspase activity assay measures activation of various caspases in a very early event of apoptosis, 2. The Annexin-V stains certain compounds which are present on the cell membrane in a more advanced event, and 3. the DNA fragmentation occurs late in the apoptotic process. The results indicate that selected inhibitors have effect in at least two different apoptosis assays.

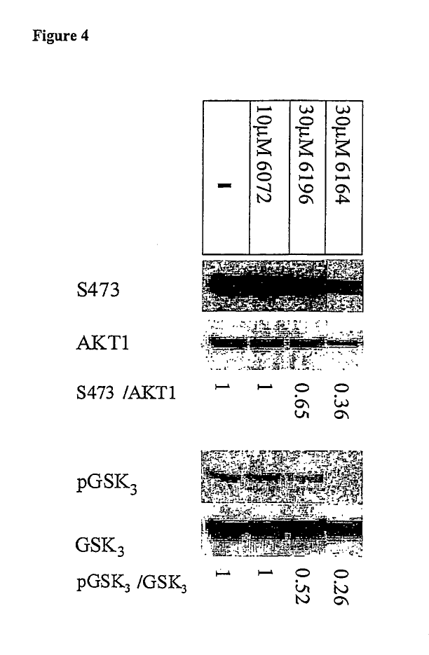

FIG. 4 discloses identification of AKT (S473) and GSK3 (S9/21) phosphorylation in LNCaP cells. Cells were treated with 30 μM of PTRs 6164, 6196 or with 10 μM of 6072 for 24 hrs and phosphorylation was measured by Western Blot analysis.

Table 6 depicts DNA fragmentation as marker for apoptosis in LNCaP cells and caspases activity in Jurkat cells: LNCap cells were treated with 30 μM of PTR 6198, 6196, 6164 or 6244 for 72 hrs. Jurkat cells were treated with 15 μM of PTR 6198, 6196, 6164 or with 25 μM of PTR 6244 for 24 hrs. Apoptosis was measured by FACS analysis of DNA fragmentation measurement in LNCaP cells and caspase activity in Jurkat, as detailed in materials and methods.

TABLE 6

| | LNCaP cells | | Jurkart cells | |
|---|---|---|---|---|
| | Concentration | | Concentration | |
| PTR | (μM) | Apoptosis % | (μM) | Apoptosis % |
| 6196 | 30 | 17 | 15 | 48 |
| 6198 | 30 | 32 | 15 | 47 |
| 6244 | 30 | 38 | 25 | 74 |
| 6164 | 30 | 40 | 15 | 78 |

### Example 8

Further Characterization of PTR 6164 as Cell-Permeable, Serum Stable Inhibitor of PKB

PTR 6164, which was now discovered as active PKB inhibitor, has been further characterized in several additional assays. The emerging picture is that this compound is a potent, selective, serum stable and cell permeable PKB inhibitor, that is a promising candidate to further development to an anti cancer drug. It induces cell death in cancer cells, apoptosis in three different assays and decreases the phosphorylation of GSK3 and FKHR.

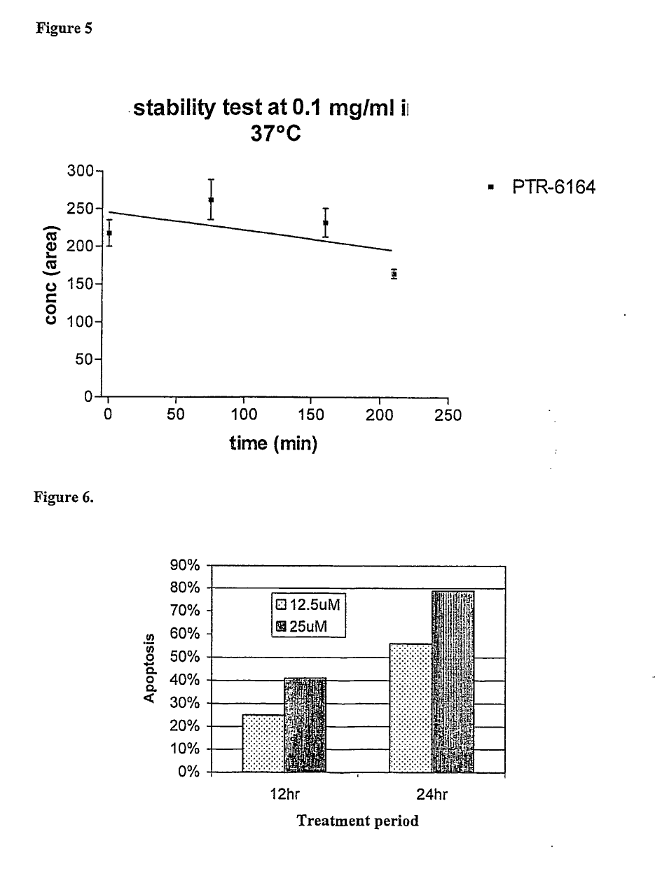

FIG. 5 show the results of biostability of PTR-6164 in mouse plasma at 37° C. PTR 6164 0.5 mg were dissolved in 50 μL of DMSO and diluted in 450 of water. 10 μl of this solution were added to 90 μl of mouse (Balb C) plasma in different times, triplicate for each time, for incubation in 37OC. After incubation period the samples were frozen over night. Peptide quantity was determined by HPLC-MS directly from the plasma after dilution 1:5 in water. Each sample was injected twice using the following specifications:

Column: pep85 C₁₈ Zobrax 2.1*50 mm

Eluent: MeCN:water (+0.1%TFA) 8:2, the peptide appears at R_{T} of 2.1 minutes.

Detector: SRM, MS in splitless mode.

Run time 6 minutes.

FIG. 6 show apoptosis induced in Jurkat cells using Annexin-V staining. Cells were treated with 12.5 μM or 25 μM of PTR 6164 for 12 and 24 hrs, and Time/dose dependent apoptosis was measured by Annexin-V staining and FACS analysis.

### Example 9

Cell Growth Inhibition Induced by our PKB Inhibitors in Cancer Cells vs. Normal Blood Cells.

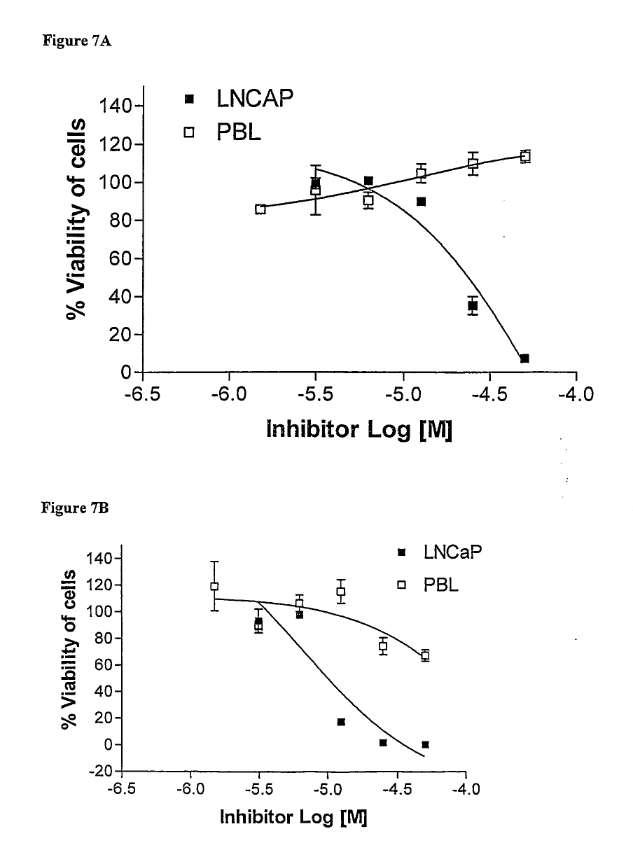

In order to evaluate the safety of the inhibitors, induced cancer (prostate LNCaP line) cell death was compared with induced normal blood cells death. It could be seen in FIG. 7 that the peptide conjugates PTR 6164 (A) and PTR 6260 (B) cause death in cancer cells at 7-11 μM, but are safe to normal cells at >50 μM. In contrast, small molecules (PTR 6074) are only 3 times more toxic to cancer than to normal cells.

### Example 10

In Vivo Studies

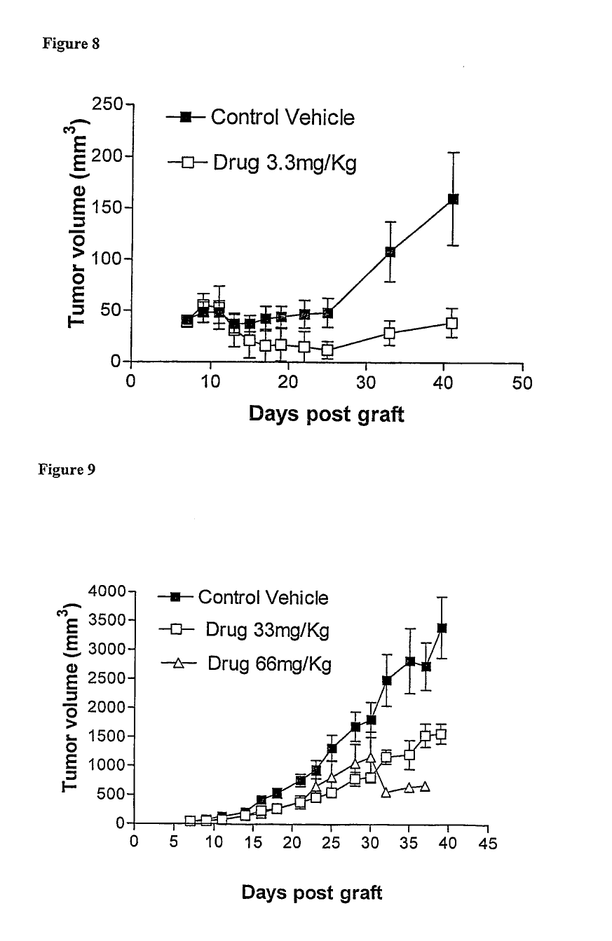

Selected active conjugates, including the lead compound PTR 6164, were tested in assay C1 above for determination of the maximal tolerated dose (MTD) for its use. The MTD value found for PTR 6164 was above 130 mg/kg, which is considered a very safe value. The high safety of PTR 6164 allows a very large therapeutic window. The conjugates were further tested in vivo in efficacy studies as described in C2 above:
a. The peptides were injected every other day, at the tumor area. After 3 injections, an indicative tendency of reduction in tumor size was observed. In the second week, the reduction continues, and in the group treated by the high dose of PTR 6164, four out of five animals showed complete regression of the tumor as described in FIG. 8.

Although the peptide dose was 3.3 mg/kg the MTD for this compound is 40 times higher, indicating a high therapeutic index.
b. Two concentrations were tested, 66 mg/kg and 33 mg/kg, which are 0.5 and 0.25 of the MTD. The relatively high doses were selected because we had no previous knowledge on the pharmacokinetics of the compound, and we wanted to obtain maximum effect, although we expected that toxic effects might occur as a result of the high dosing.

The compounds were dissolved in a solution of 10% DMSO/90% cyclodextrin in physiological saline. Six groups of 9 mice in each were tested. Two groups received the compound i.v, every 48 hours, and two groups received it daily, by i.p. injection. Treatment started at 7 days post graft, lasted for 21 days, followed by a 14-day observation period. The experiment consisted four experimental groups as in table 7:

TABLE 7

| | | | Dose | |
|---|---|---|---|---|
| Group | Treatment | Administration Route | (mg/kg) | Schedule |
| 1 | Vehicle | Intraperitoneal (IP) | 0 | 1x daily for 3 wks |
| 2 | Peptide | Intraperitoneal (IP) | 20 | 1x daily for 3 wks |
| 3 | Vehicle | Intraperitoneal (IP) | 0 | 2x daily for 3 wks |
| 4 | Peptide | Intraperitoneal (IP) | 10 | 2x daily for 3 wks |

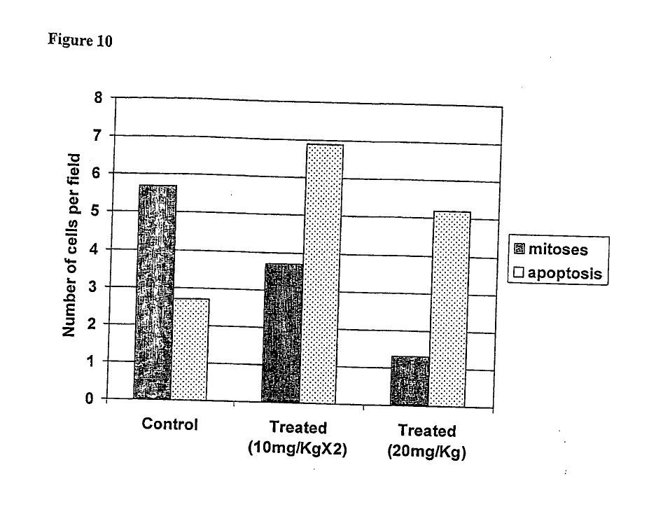

The results indicate that groups that received the injections every 48 hours showed no effect on the tumor size. However, the groups that received daily injections, showed significant inhibition of tumor growth. Interestingly, both doses, 66 mg/kg and 33 mg/kg showed the same effect, implying that it might be possible to go even lower with the concentration without loosing efficacy. Most importantly, the experiment proved that the compound could be efficiently distributed through the blood stream into the tumor, which for peptide-based drug is an extremely important observation. FIG. 9 depicts the graph of tumor size in the control and treated animals in the two i.p. treated groups, and FIG. 10 describes the effect of PTR 6164 on apoptosis and mitosis in stained tumor sections from nude mice PC3 bearing xenografts. It is clear that the effect of the inhibitors was very significant, despite the aggressiveness of the tumors. The preliminary experiments with this compound proved the fact that peptide-based compounds of the general structure of 6164, can efficiently and effectively be administered systemically.

### Example 11

Optimization of PTR 6164

An optimization process was performed for the prototype compound PTR 6164, and new compounds having the general structure of PTR 6164, with structural peptidomimetic modifications were synthesized and screened for inhibition of protein kinase activity. The sequences and the activity results are presented in table 8. It was surprisingly found that addition of Lys₂₋₄ to PTR 6164 contributes to the in vitro activity of the compounds. In particular few of the new compounds were found to be about five times more potent than PTR 6164, in all cellular assays, and are remarkably more selective.

TABLE 8

| | | | IC₅₀ μM | | | |
|---|---|---|---|---|---|---|
| | | | In vitro | | | |
| | | | kinase | | | |
| | SEQ ID | | assay | | Cells | |
| PTR # | NO: | Sequence | PKB | PKA | LNCaP | PBLs |
| 6962 | 53 | Cholesteryl-O—CO-(DLys)₈-Arg-Pro-Arg- | 0.1 | 10-30 | 2 | 12 |
| | | Nva-Tyr-Dap-Hol-NH₂ | | | | |
| 6294 | 54 | Cholesteryl-O—CO-Gly-Arg-Pro-Arg-Nva- | 1 | 3-10 | 12 | 50 |
| | | Tyr-Dap-Hol-NH₂ | | | | |
| 6296 | 55 | Cholesteryl-O—CO-betaAla-Arg-Pro-Arg- | 2 | 3-10 | 12 | >25 |
| | | Nva-Tyr-Dap-Hol-NH₂ | | | | |
| 6298 | 56 | Cholesteryl-O—CO-GABA-Arg-Pro-Arg- | 2 | 3-10 | 12-25 | >50 |
| | | Nva-Tyr-Dap-Hol-NH₂ | | | | |
| 6300 | 57 | Cholesteryl-O—CO-Arg-Pro-Arg-Orn- | 0.3-1 | 0.3 | 15 | 12 |
| | | Glu(aminopropylmercaptoisoquinoline)- | | | | |
| | | Ser-Phe-NH₂ | | | | |
| 6304 | 58 | Cholesteryl-O—CO-Arg-Pro-Arg-Nva-Tyr- | 0.5 | 1-3 | 25 | ND |
| | | Ser(Me)-Hol-NH₂ | | | | |
| 6306 | 59 | Cholesteryl-O—CO-Gly-Arg-Pro-Arg-Nva- | 0.5 | ND | 20 | ND |
| | | Tyr-Ser(Me)-Hol-NH₂ | | | | |
| 6308 | 60 | Cholesteryl-O—CO-bAla-Arg-Pro-Arg- | 0.5 | ND | 25 | ND |
| | | Nva-Tyr-Ser(Me)-Hol-NH₂ | | | | |
| 6310 | 61 | Cholesteryl-O—CO-GABA-Arg-Pro-Arg- | ND | ND | 25 | ND |
| | | Nva-Tyr-Ser(Me)-Hol-NH₂ | | | | |
| 6312 | 62 | H-(DLys)₈-Arg-Pro-Arg-Nva-Tyr-Dap- | 0.1 | 10-30 | 6 | 12 |
| | | Hol-Lys(CO—O-Cholesteryl)-NH₂ | | | | |
| 6316 | 63 | H-Arg-Gln-Ile-Lys-Ile-Trp-Phe-Gln-Asn- | 0.1 | 5 | 6 | 20 |
| | | Arg-Arg-Met-Lys-Trp-Lys-Lys-Arg-Pro- | | | | |
| | | Arg-Nva-Tyr-Dap-Hol-Lys(CO—O- | | | | |
| | | Cholesteryl)-NH₂ | | | | |
| 6318 | 21 | Cholesteryl-O—CO-(DLys)₂-Arg-Pro-Arg- | 0.2 | 10-30 | 3 | 20 |
| | | Nva-Tyr-Dap-Hol-NH₂ | | | | |
| 6320 | 10 | Cholesteryl-O—CO-(DLys)₃-Arg-Pro-Arg- | 0.2 | 10-30 | 2 | 30 |
| | | Nva-Tyr-Dap-Hol-NH₂ | | | | |
| 6322 | 12 | Cholesteryl-O—CO-(DLys)₄-Arg-Pro-Arg- | 0.1 | 10-30 | 3 | 30 |
| | | Nva-Tyr-Dap-Hol-NH₂ | | | | |
| 6324 | 64 | Cholesteryl-O—CO-(DLys)₅-Arg-Pro-Arg- | 0.1 | 10-30 | 2 | 12 |
| | | Nva-Tyr-Dap-Hol-NH₂ | | | | |
| 6326 | 65 | Cholesteryl-O—CO-(DLys)₆-Arg-Pro-Arg- | 0.1 | ND | 3 | 10 |
| | | Nva-Tyr-Dap-Hol-NH₂ | | | | |
| 6328 | 66 | Cholesteryl-O—CO-(Gly)₂-Arg-Pro-Arg- | 1.0 | ND | 12 | ND |
| | | Nva-Tyr-Dap-Hol-NH₂ | | | | |
| 6330 | 67 | Cholesteryl-O—CO-(Gly)₃-Arg-Pro-Arg- | 1.0 | 3-10 | 6 | 25-50 |
| | | Nva-Tyr-Dap-Hol-NH₂ | | | | |
| 6332 | 68 | Cholesteryl-O—CO-(Gly)₄-Arg-Pro-Arg- | 1.0 | ND | 10 | >50 |
| | | Nva-Tyr-Dap-Hol-NH₂ | | | | |
| 6334 | 69 | Cholesteryl-O—CO-(Gly)₅-Arg-Pro-Arg- | 1.0 | ND | 10 | ND |
| | | Nva-Tyr-Dap-Hol-NH₂ | | | | |
| 6336 | 70 | Cholesteryl-O—CO-(Gly)₆-Arg-Pro-Arg- | 1.0 | ND | 10 | ND |
| | | Nva-Tyr-Dap-Hol-NH₂ | | | | |
| 6338 | 13 | Cholesteryl-O—CO-(DLys)₆-Arg-Pro-Arg- | 0.1 | ND | 1.5-3 | 10 |
| | | Nva-Tyr-Ser(Me)-Hol-NH2 | | | | |
| 6344 | 11 | Cholesteryl-O—CO-(Lys)₃-Arg-Pro-Arg- | 0.3 | ND | 1.5-3 | 30 |
| | | Nva-Tyr-Dap-Hol-NH₂ | | | | |
| 6346 | 14 | Cholesteryl-O—CO-Arg-Pro-Arg-Nva-Tyr- | 0.2 | ND | 3-6 | 25 |
| | | Dap-Hol-(DLys)₃-NH₂ | | | | |
| 6348 | 15 | Cholesteryl-O—CO-(DLys)₃-Arg-Pro-Arg- | 0.2 | ND | 3 | 20 |
| | | Nva-Tyr-Ser(Me)-Hol-NH₂ | | | | |
| 6350 | 71 | Decanoyl-Arg-Pro-Arg-Nva-Tyr-Dap-Hol- | ND | ND | >50 | ND |
| | | NH₂ | | | | |
| 6352 | 16 | Cholesteryl-O—CO-(Lys)₃-Arg-Pro-Arg- | 0.2 | ND | 2 | 50 |
| | | Nva-Tyr-Dap-Hol-OH | | | | |
| 6354 | 72 | Cholesteryl-O—CO-(CH2)₂-CO-Arg-Pro- | ND | ND | 12 | ND |
| | | Arg-Nva-Tyr-Dap-Hol-NH₂ | | | | |
| 6356 | 17 | Cholesteryl-O—CO-(CH2)₂-CO-(DLys)₃- | 0.17 | ND | 2 | 25 |
| | | Arg-Pro-Arg-Nva-Tyr-Dap-Hol-NH₂ | | | | |
| 6358 | 73 | Sperminyl-CO-Arg-Pro-Arg-Nva-Tyr- | ND | ND | >50 | ND |
| | | Dap-Hol-NH₂ | | | | |
| 6360 | 74 | Z-2-AcO-cinnamoyl-Arg-Pro-Arg-Nva- | ND | ND | >50 | ND |
| | | Tyr-Dap-Hol-NH₂ | | | | |
| 6362 | 75 | Z-2-AcO-cinnamoyl-(DLys)₃-Arg-Pro- | ND | ND | >50 | ND |
| | | Arg-Nva-Tyr-Dap-Hol-NH₂ | | | | |
| 6364 | 76 | NH2—(CH2)₃—NH—(CH2)₄—NH—(CH2)₃—NH—CH2—CO- | ND | ND | >50 | ND |
| | | Arg-Pro-Arg-Nva-Tyr-Dap-Hol- | | | | |
| | | NH₂ (Spermine derivative) | | | | |
| 6366 | 77 | H-His-Pro-Arg-Nva-Tyr-Dap-Hol-NH₂ | >10 | ND | >50 | ND |
| 6368 | 78 | H-Arg-Pro-His-Nva-Tyr-Dap-Hol-NH₂ | >10 | ND | ND | ND |
| 6370 | 79 | H-His-Pro-His-Nva-Tyr-Dap-Hol-NH₂ | >10 | ND | ND | ND |
| 6372 | 80 | H-Arg-Pro-Orn-Nva-Tyr-Dap-Hol-NH₂ | >10 | ND | ND | ND |
| 6374 | 81 | Cholesteryl-O—CO-His-Pro-Arg-Nva-Tyr- | 1.4 | ND | 25 | ND |
| | | Dap-Hol-NH₂ | | | | |
| 6376 | 82 | Cholesteryl-O—CO-Arg-Pro-His-Nva-Tyr- | 2 | ND | ND | ND |
| | | Dap-Hol-NH₂ | | | | |
| 6380 | 83 | H-Orn-Pro-Arg-Nva-Tyr-Dap-Hol-NH₂ | >10 | ND | >50 | ND |
| 6382 | 18 | Cholesteryl-O—CO-Orn-Pro-Arg-Nva-Tyr- | 0.36 | ND | 10 | ND |
| | | Dap-Hol-NH₂ | | | | |
| 6384 | 84 | H-Orn-Pro-Orn-Nva-Tyr-Dap-Hol-NH2 | >10 | ND | ND | ND |
| 6386 | 85 | Cholesteryl-O—CO-Orn-Pro-Orn-Nva-Tyr- | 2 | ND | ND | ND |
| | | Dap-Hol-NH₂ | | | | |
| 6388 | 86 | Cholesteryl-O—CO-Arg-Pro-Orn-Nva-Tyr- | 2 | ND | ND | ND |
| | | Dap-Hol-NH₂ | | | | |
| 6390 | 87 | Cholesteryl-O—CO-Arg-Pro-Arg-Nva-Tyr- | 2 | ND | 8.8 | 50 |
| | | Dap-OH | | | | |
| 6392 | 88 | Coumarin-3-oyl-Arg-Pro-Arg-Nva-Tyr- | 5 | ND | ND | ND |
| | | Dap-Hol-NH₂ | | | | |
| 6394 | 89 | HO—CO—(CH2)₂—CO-(DLys)₃-Arg-Pro- | 10 | ND | >50 | ND |
| | | Arg-Nva-Tyr-Dap-Hol-NH₂ | | | | |
| 6396 | 20 | Vitamin E-CO—(CH2)₂—CO-(DLys)₃-Arg- | 0.2 | ND | 10 | ND |
| | | Pro-Arg-Nva-Tyr-Dap-Hol-NH2 | | | | |
| 6398 | 90 | Cholesteryl-O—CO-(DLys)₃-Orn-Pro-Arg- | 0.3 | ND | 10 | ND |
| | | Nva-Tyr-Dap-Hol-NH₂ | | | | |
| 6400 | 19 | Cholesteryl-O—CO-(DLys)₃-Lys-Pro-Arg- | 0.3 | ND | 3 | ND |
| | | Nva-Tyr-Dap-Hol-NH₂ | | | | |
| 6402 | 91 | Cholesteryl-O—CO-(DLys)₃-Arg-Pro-Arg- | 0.5 | ND | 3 | ND |
| | | Nva-Tyr-Dap-Hol-Lys(FITC)-NH₂ | | | | |

A compound was considered active (shaded background in the table) when activity was observed in cancer cells and not in normal cells (PBLs IC₅₀/LNCaP IC₅₀ >5), and when the PKB inhibitory activity is 0.5 μM or higher and the PKA inhibitory activity at least 3 times lower.

### Example 12

Synthesis of PTR 6320

Cholesteryl-O—CO—(DLys)₃-Arg-Pro-Arg-Nva-Tyr-Dap-Hol-NH₂ (SEQ ID NO:10)

One gram of Rink amide MBHA resin (0.64 mmol/g), was swelled for 3 h with N-methylpyrrolidonene (NMP) in a reaction vessel equipped with a sintered glass bottom and placed on a shaker. All Fmoc protecting groups were removed by reaction with 25% piperidine in NMP (2 times 15 minutes, 10 ml each) followed by NMP wash (5 times two minutes, 15 ml each). Fmoc removal was monitored by ninhydrin test. The first amino acid was coupled to the resin by using 3 eq of the Fmoc protected amino acid (Fmoc-Hol-OH)+3 eq PyBroP+6 eq of DIEA in 7 ml NMP, reaction time, 1.5 h. The couplings of the other Fmoc protected amino acids [Fmoc-Dap(Boc)—OH, Fmoc-Tyr(tBu)—OH, Fmoc-Nva-OH, Fmoc-Arg(Pbf)—OH and Fmoc-DLys (Boc)—OH] were carried out using 3 eq (1.92 mmol) of the Fmoc amino acid+PyBrop (3 equivalents, 1.92 mmol)+DIEA (6 equivalents, 3.84 mmol) in NMP (7 ml) for 1 hour at room temperature (or alternatively using 3 eq Fmoc-AA-OH+3 eq HOBT+3 eq DIC in DMF for 1 h at room temperature). Reaction completion was monitored by the qualitative ninhydrin test (Kaiser test). After each coupling, the peptide-resin was washed with NMP (5 times with 15 ml NMP, 2 minutes each). The coupling of cholesterol to the N-terminal DLys was carried out by addition of 15 ml solution of dioxane: 1,3-dichloropropane 1:1 containing cholesterol (5 eq)+BTC (1.66 eq)+collidine (15 eq), coupling time 1.5 h at 60° C.

In the end of the assembly, the peptide resin was washed with CH₂Cl₂ and dried under reduced pressure then cleaved from the resin by reaction with 25 ml TFA solution containing 7% TIS (tri-isopropyl-silane), 23% CH₂Cl₂ , at 0° C. for 10 minutes and 1 h at room temperature under argon. The mixture was filtered and the resin was washed with a small volume of 70% TFA in CH₂Cl₂. The filtrate was placed in a rotary evaporator and all the volatile components were removed. An oily product was obtained. It was triturated with ether and the ether decanted, three times. A white powder was obtained. The crude product was dried under reduced pressure (65% yield of crude peptide).

### Example 13

Additional Characterization of PTR 6320 and 6344

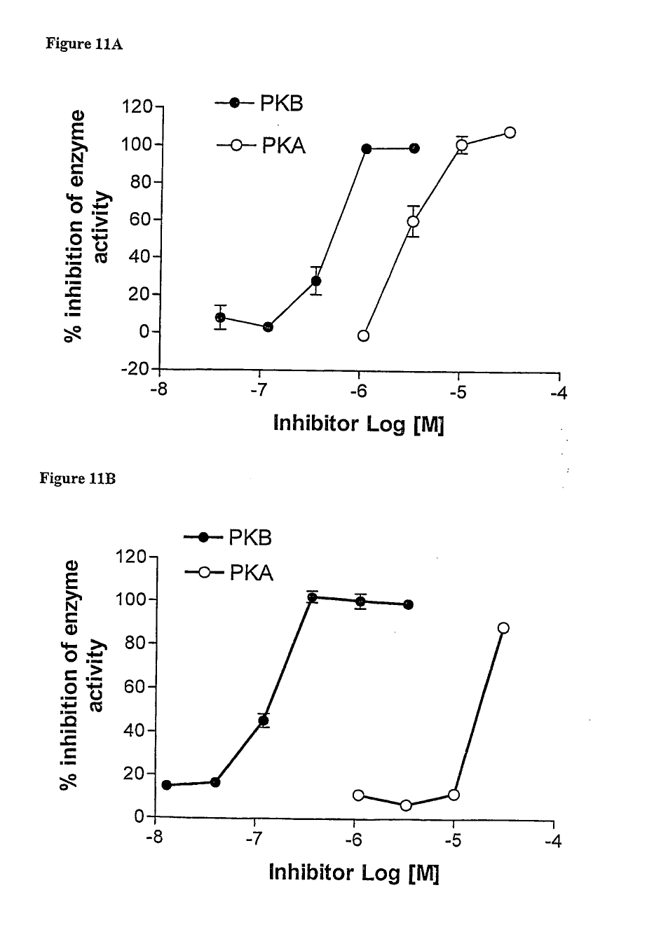

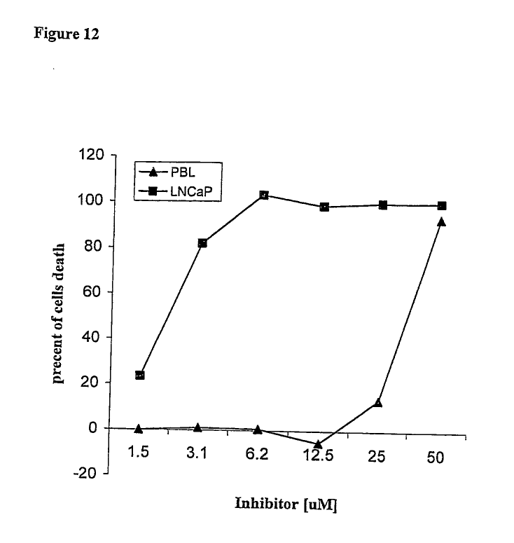

In a series of in vitro assays it was found that PTR 6320 and 6344 induce cell death and apoptosis at 1-2 μM in prostate cancer cell lines, but are safe to normal cells at 40 μM. They induce apoptosis in cancer cells at 3 μM and show inhibition of downstream substrates of PKB by western blot at 3-5 μM. FIG. 11 shows the improved in vitro selectivity (about 20 times) of PTR 6320 in comparison to PTR 6164 in inhibition of protein kinase B vs. protein kinase A activity. Table 9 represents the cell death induced by the protein kinase inhibitors in prostate cancer cell lines vs. normal cells. The magnitude of cell death induced is in direct correlation with the levels of activated PKB in the various cells, as depicted in FIG. 12. Optimized leads PTR 6320 and 6344 show five times better efficacy and improved selectivity than the prototype molecule PTR 6164. Levels are in direct correlation with the magnitude of cell death induced by our inhibitors, as depicted in table 9.

TABLE 9

| | | LNCaP | PC3 | MCF10 | PBLs |
|---|---|---|---|---|---|
| PTR No: | SEQ ID NO: | IC50 μM | | | |
| 6164 | 22 | 10 | 25 | 40 | 50 |
| 6320 | 10 | 2 | 12 | 40 | 40 |
| 6344 | 11 | 2 | 12 | 25 | 40 |

TABLE 10
describes the IC₅₀ values of PTR 6164 and its optimized compounds
in human breast adenocarcinoma cell line MDA-468 and in human renal
cell line 786-O

| | |
|---|---|
| MDA-468 | 786-O |

| | | |
|---|---|---|
| PTR | SEQ ID NO: | IC₅₀ μM |

| | | | |
|---|---|---|---|
| 6164 | 22 | 30 | 20 |
| 6292 | 53 | 6 | 2 |
| 6312 | 62 | 10 | 8 |
| 6318 | 21 | 20 | 12 |
| 6320 | 10 | 10 | 10 |
| 6322 | 12 | 10 | 6 |
| 6324 | 64 | 10 | 5 |
| 6326 | 65 | 10 | 3 |
| 6330 | 67 | 20 | 12 |
| 6338 | 13 | 4 | 3 |
| 6344 | 11 | 12 | 6 |
| 6346 | 14 | 20 | 10 |
| 6348 | 15 | 10 | 6 |

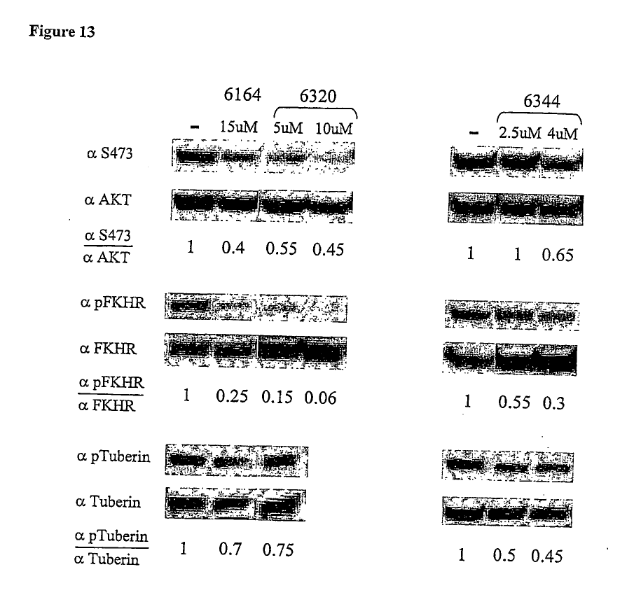

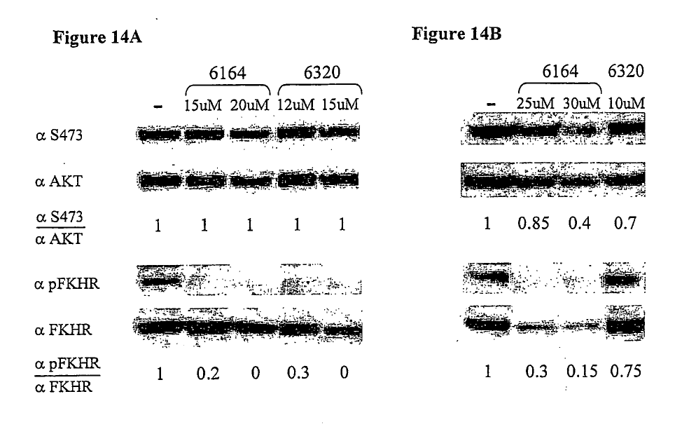

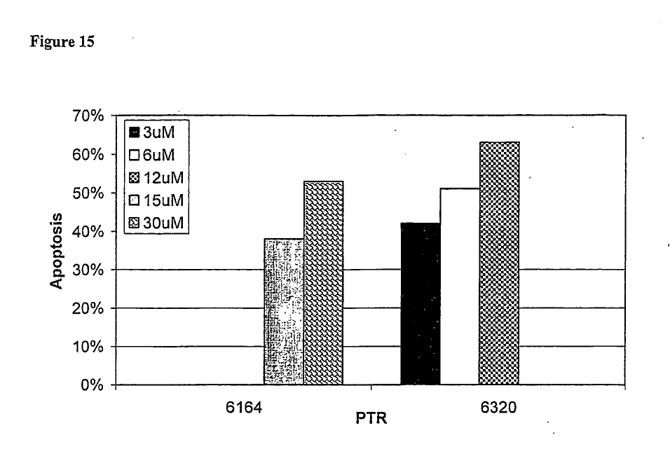

FIG. 13 describes western blot analysis of AKT and FKHR phosphorylation in LNCaP cells treated with PTR 6164, 6320 and 6344. FIG. 14 describes western blot analysis of AKT and FKHR phosphorylation in 786-O (A) and MDA468 (B) cells treated with PTR 6164 and 6320, and FIG. 15 depicts Induction of apoptosis in prostate cancer cells (LNCaP) by PTR 6164 and PTR 6320 as measured by caspase activity.

In addition, PTR 6320 was analyzed by LC-MS for in vitro metabolism in hepatic carcinoma cells. The results indicate initial drug metabolism after 1 hour and advanced metabolism after 24 hours. Hep G2 cells were incubated with 30 μM of PTR 6320 for 6 hours and peptides were extracted from the supernatants and the pellets of the cells. Samples were analyzed by LC-MS using pep 24 C18 Vydac 1.0*150 mm column in eluent gradient of MeCN:water (+0.1%TFA) with MS ESI detector in splitless mode. The results show that after 6 hours there is still substantial amount of undegraded peptide with one major metabolite of PTR 6320, which is des Tyr-Dap-Hol from the C terminus of the peptide. This indicates slow degradation of PTR 6320 in hepatic cells implying that peptide may be retained in the system enough time for efficient distribution.

Results of apoptosis and mitosis in tumor sections from treated vs. untreated animals (i.p. treatment using 33 mg/kg of PTR 6164), are presented in table 11. Three tumors from each group were studies and a clear trend of increased apoptosis is observed in the treated tumors, implying induction of apoptosis through inhibition of PKB. Tumors were collected after the 14 days observation period in which no treatment was given, indicating long-term effect. The ratio between apoptosis to mitoses in tumor sections express the viability of the tumor. In a viable tissue mitotic process are dominant and apoptosis is minor while in dying tissue this ratio is reversed. When there is regression in tumor growth, there are more apoptotic cells than mitotic cells, as in tumors from treated mice.

TABLE 11

| | Treated | Control |
|---|---|---|
| Mitoses | 3.4 | 6 |
| Apoptosis | 5.6 | 3.6 |

### Example 14

Combination Treatment with PTR 6320 and other Chemotherapeutic Agents

The chemotherapeutic agents used are: Mitoxantrone hydrochloride (Novantrone, Wyeth Lederle S.p.A. Catania, Italy) supplied as a sterile aqueous solution of 3.8 mM. Doxorubicin, Etoposide and Vinblastine sulfate salt (Sigma) were dissolved in 100% DMSO to prepare stock solutions at concentration of 10 mM. Docetaxel (Taxpter, Aventis) stock solution of 11.6 mM in polysorbate 80. These agents were diluted in medium just before use for in vitro studies.

Growth inhibition assay was preformed as described above in the present of six concentration of chemotherapeutic agent (above and under their IC₅₀ values) with or without PTR 6320. Cells were incubated for five days in the present of the drugs followed by fixation and staining of cells with methylene blue. IC₅₀ values were calculated as described above for each chemotherapeutic agents alone and in the present of PTR 6320.

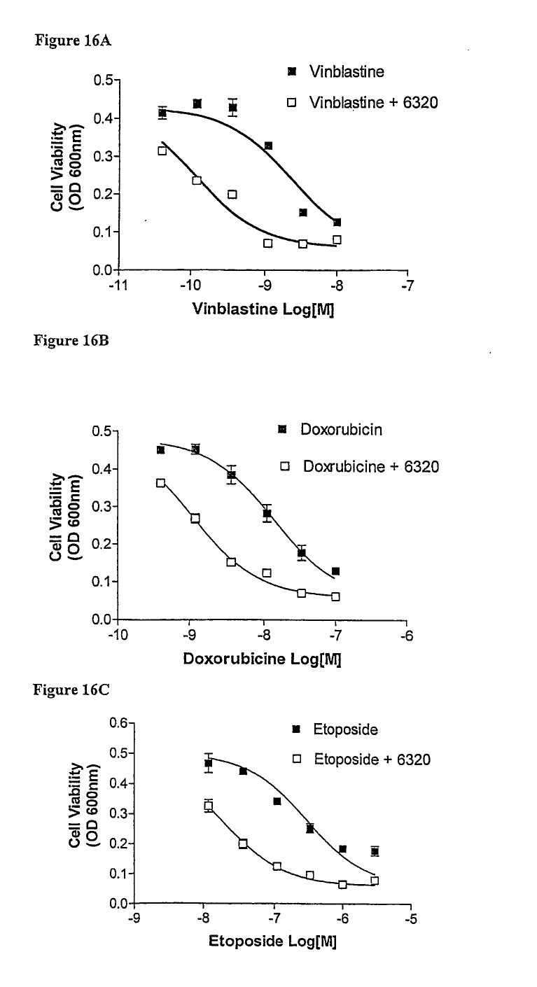

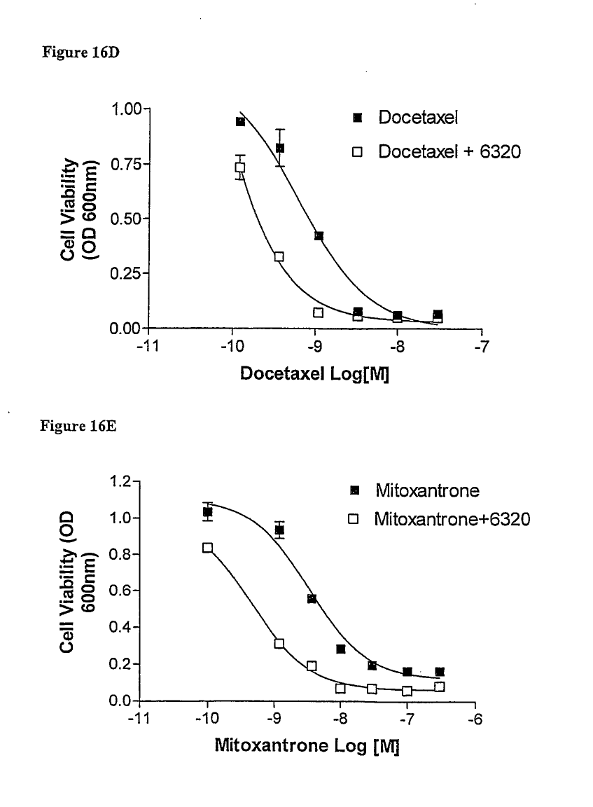

Synergistic effects in cell death of prostate cancer cell lines, using combination treatment of protein kinase inhibitors of the present invention together with several commercial chemotherapy agents are shown in FIGS. 16 A-E. A 10-20-fold improvement in the IC₅₀ of each drug is observed when combined with 1.5 μM of PTR 6320. The results are summarized in table 12.

TABLE 12

| | IC₅₀ (nM) | |
|---|---|---|
| Drug | — | +PTR 6320 |
| Vinblastine | 2.2 | 0.1 |
| Doxorubicine | 13 | 1.1 |
| Etoposide | 300 | 17 |
| Docetaxel | 0.6 | 0.03 |
| Mitoxantrone | 3.4 | ˜0.36 |

While the present invention has been described for certain preferred embodiments and examples it will be appreciated by the skilled artisan that many variations and modifications may be performed to optimize the activities of the peptides and analogs of the invention. The examples are to be construed as non-limitative and serve only for illustrative purposes of the principles disclosed according to the present invention, the scope of which is defined by the claims which follow.

1. A protein kinase inhibitor comprising a molecule having at least a first moiety competent for penetration of the molecule into cells, and a second moiety having a protein kinase inhibiting effect within the cells, the first moiety being linked to the second moiety through a direct bond or a spacer.

2. The protein kinase inhibitor of claim 1 which is selective for protein kinase B (“PKB”).

3. The protein kinase inhibitor of claim 2 wherein the second moiety is a peptide or a peptidomimetic comprising a PKB substrate or a PKB substrate mimetic.

4. The protein kinase inhibitor of claim 3 wherein the PKB substrate is glycogen synthase kinase 3 (GSK3).

5. The protein kinase inhibitor of claim 1 wherein the second moiety is a peptide of 5-20 amino acids or a peptidomimetic.

6. The protein kinase inhibitor of claim 1 wherein the first moiety and the second part are linked directly via a covalent bond.

7. The protein kinase inhibitor of claim 6, wherein the first moiety and the second part are linked directly via an amide bond.

8. The protein kinase inhibitor of claim 1 wherein the first moiety and the second part are linked through a spacer.

9. The protein kinase inhibitor of claim 8, wherein the first moiety and the second part are linked through a spacer comprising at least one amino acid residue.

10. The protein kinase inhibitor of claim 1 wherein the first moiety is linked to the amino terminus or carboxy terminus of a protein kinase inhibitory peptide moiety.

11. The protein kinase inhibitor of claim 1 further comprising an ATP mimetic moiety.

12. The protein kinase inhibitor of claim 1 wherein the second moiety comprises a peptide of Formula I (SEQ ID NO:2):
M-X₁-Pro-Arg-X₄-X₅-X₆-X₇
wherein,
M is absent or is selected from D- or L-Lys₂₋₄;
X₁ is Arg, Lys, Orn or Dab;
X₄ is Nva, Leu, Ile, Abu or Orn;
X₅ is Tyr, Gly, GlyNH₂, Ser(Me), Glu, or Glu(NH—(CH2)2-NH—SO₂-isoquinoline);
X₆ is Dap, Abu, GlyNH2, Ser(Me), Gly, Ala or Ser; and
X₇ is an aromatic or an aliphatic bulky residue;
and analog, salt or functional derivative thereof,
or a peptide of Formula II (SEQ ID NO:3):
M-Arg-Pro-Arg-X₄-X₅-X₆-X₇
wherein,
M is DLys₃ or Lys₃;
X₄ is Nva, Leu, Ile, Abu or Orn;
X₅ is Tyr, Gly, GlyNH₂, Ser(Me), Glu, or Glu(NH—(CH2)2-NH—SO₂-isoquinoline);
X₆ is Dap, Abu, GlyNH2, Ser(Me), Gly, Ala or Ser; and
X₇ is an aromatic or an aliphatic bulky residue;
and analog, salt or functional derivative thereof.

13. The protein kinase inhibitor of claim 12 comprising a peptide selected from the group consisting of:
DLys-DLys-DLys-Arg-Pro-Arg-Nva-Tyr-Dap-Hol (SEQ ID NO:5);
Lys-Lys-Lys-Arg-Pro-Arg-Nva-Tyr-Dap-Hol (SEQ ID NO:6);
Arg-Pro-Arg-Nva-Tyr-Dap-Hol (SEQ ID NO:7);
Arg-Pro-Arg-Orn-Glu-(NH—(CH2)2-NH—SO₂-Isoquinoline)Ser-Phe (SEQ ID NO:8); and
Arg-Pro-Arg-Nva-Tyr-Ala-Hol (SEQ ID NO:9).

14. The protein kinase inhibitor of claim 12 wherein the cell-permeability moiety is selected from the group consisting of: cholesterol, (DLys)₂₋₁₀, (Lys)₂₋₁₀, vitamin E, Arg-Gln-Ile-Lys-Ile-Trp-Phe-Gln-Asn-Arg-Arg-Met-Lys-Trp-Lys-Lys, Ahx6-DArg-DArg-DArg-DArg-DGln-Arg-DLys-DLys-DArg, and (DArg)₇₋₉; and Z absent or is selected from carbamate and Gly.

15. The protein kinase inhibitor of claim 1 comprising a peptide conjugate of Formula III:
Y-Z-Arg-Pro-Argg-Nva-Tyr-X₆-Hol (SEQ ID NO:4)
wherein X₆ is Dap or Ala;
Y is a cell-permeability moiety; and
Z is a spacer or a bond connecting Y to the peptide.

16. The protein kinase inhibitor of claim 15 wherein Y is selected from the group consisting of: cholesterol, (DLys)₂₋₁₀, (Lys)₂₋₁₀, vitamin E, Arg-Gln-Ile-Lys-Ile-Trp-Phe-Gln-Asn-Arg-Arg-Met-Lys-Trp-Lys-Lys, Ahx6-DArg-DArg-DArg-DArg-DGln-Arg-DLys-DLys-DArg, and (DArg)₇₋₉; and Z absent or is selected from carbamate and Gly.

17. A protein kinase inhibitor selected from the group consisting of:
Cholesteryl-O—CO-DLys-DLys-DLys-Arg-Pro-Arg-Nva-Tyr-Dap-Hol-NH₂ (SEQ ID NO:10);
Cholesteryl-O—CO-Lys-Lys-Lys-Arg-Pro-Arg-Nva-Tyr-Dap-Hol-NH₂ (SEQ ID NO:11);
Cholesteryl-O—CO-(DLys)₄-Arg-Pro-Arg-Nva-Tyr-Dap-Hol-NH₂ (SEQ ID NO:12 );
Cholesteryl-O—CO-(DLys)₆-Arg-Pro-Arg-Nva-Tyr-Ser(Me)-Hol-NH2 (SEQ ID NO:13);
Cholesteryl-O—CO-Arg-Pro-Arg-Nva-Tyr-Dap-Hol-(DLys)₃-NH₂ (SEQ ID NO:14);
Cholesteryl-O—CO-(DLys)₃-Arg-Pro-Arg-Nva-Tyr-Ser(Me)-Hol-NH₂ (SEQ ID NO:15);
Cholesteryl-O—CO-( Lys)₃-Arg-Pro-Arg-Nva-Tyr-Dap-Hol-OH (SEQ ID NO:16);
Cholesteryl-O—CO—(CH2)₂-CO-(DLys)₃-Arg-Pro-Arg-Nva-Tyr-Dap-Hol-NH₂ (SEQ ID NO:17);
Cholesteryl-O—CO-Orn-Pro-Arg-Nva-Tyr-Dap-Hol-NH₂ (SEQ ID NO:18);
Cholesteryl-O—CO-(DLys)₃-Lys-Pro-Arg-Nva-Tyr-Dap-Hol-NH₂ (SEQ ID NO:19);
Vitamin E-CO—(CH2)₂-CO-(DLys)₃-Arg-Pro-Arg-Nva-Tyr-Dap-Hol-NH₂ (SEQ ID NO:20);
Cholesteryl-O—CO-(DLys)₂-Arg-Pro-Arg-Nva-Tyr-Dap-Hol-NH₂ (SEQ ID NO:21);
Cholesteryl-O—CO-Arg-Pro-Arg-Nva-Tyr-Dap-Hol-NH₂ (SEQ ID NO:22);
Cholesteryl-O—CO-Arg-Pro-Arg-Nva-Tyr-Ala-Hol-NH₂ (SEQ ID NO:23);
Cholesteryl-O—CO-Gly-Arg-Pro-Arg-Nva-Tyr-Ala-Hol-NH₂ (SEQ ID NO :24);
Vitamin E-Succinate-Arg-Pro-Arg-Nva-Tyr-Dap-Hol-NH₂ (SEQ ID NO:25);
H-(DArg)₉-Gly-Arg-Pro-Arg-Nva-Tyr-Ala-Hol-NH₂ (SEQ ID NO:26); and
Cholesteryl-O—CO-Arg-Pro-Arg-Orn-Glu(Aminoethylsulfonamidoisoquinoline)-Ser-Phe-NH₂ (SEQ ID NO:27).

18. A pharmaceutical composition comprising as an active ingredient a protein kinase inhibitor comprising a molecule having at least a first moiety competent for penetration of the molecule into cells, a second moiety for having a protein kinase inhibiting effect within the cells, the first moiety being joined to the second moiety through a linker, and a pharmaceutically acceptable excipient, carrier or diluent.

19. A method of treatment of a disease comprising administering to a patient in need of such treatment a therapeutically effective amount of a protein kinase inhibitor according to claim 1.

20. The method according to claim 19 wherein the protein kinase inhibitor is administered in a pharmaceutical composition that includes a pharmaceutically acceptable excipient, carrier or diluent.

21. The method according to claim 19 wherein the disease is one selected from the group comprising cancers, abnormal proliferation disease, diabetes, cardiovascular pathologies, hemorrhagic shock, obesity, inflammatory diseases, diseases of the central nervous system, and autoimmune diseases.

22. The method according to claim 21 wherein the disease is selected from the group consisting of: prostate cancer; breast cancer; ovarian cancer; colon cancer; renal cancer; melanoma and skin cancer; lung cancer; and hepatocarcinoma.

23. The method according to claim 21 wherein the disease is selected from restenosis, benign tumors, atherosclerosis, insults to body tissue due to surgery, abnormal wound healing, abnormal angiogenesis, diseases that produce fibrosis of tissue, repetitive motion disorders, disorders of tissues that are not highly vascularized, and proliferative responses associated with organ transplants.

24. A method for inhibiting tumor progression in a mammal in need thereof, comprising administering a therapeutically effective amount of the protein kinase inhibitor of claim 1 to the mammal, and co-administering a therapeutically effective amount of at least one cytotoxic agent in an amount sufficient to inhibit tumor progression in the mammal.

25. The method according to claim 24 wherein the protein kinase inhibitor is administered in a pharmaceutical composition that includes a pharmaceutically acceptable excipient, carrier or diluent and that optionally includes the at least one cytotoxic agent.
